(19) United States
(12) Patent Application Publication
(10) Pub. No.: US 2025/0221607 A1
(43) Pub. Date: Jul. 10, 2025

(54) MEDICAL SUPPORT DEVICE, ENDOSCOPE, MEDICAL SUPPORT METHOD, AND PROGRAM

(71) Applicant: FUJIFILM Corporation, Tokyo (JP)

(72) Inventor: Masaaki OOSAKE, Kanagawa (JP)

(73) Assignee: FUJIFILM Corporation, Tokyo (JP)

(21) Appl. No.: 19/094,992

(22) Filed: Mar. 30, 2025

Related U.S. Application Data
(63) Continuation of application No. PCT/JP2023/036267, filed on Oct. 4, 2023.

(30) Foreign Application Priority Data
Nov. 4, 2022 (JP) 2022-177611

(51) Int. Cl. A61B 1/00; A61B 1/04; G16H 10/60; G16H 30/40

(52) U.S. Cl. CPC A61B 1/000094; A61B 1/0005; A61B 1/04; G16H 10/60; G16H 30/40

(57) ABSTRACT

A medical support device includes a processor. The processor is configured to detect a duodenal papilla region by executing image recognition processing on an intestinal wall image obtained by a camera provided in an endoscopic scope imaging an intestinal wall of a duodenum; display the intestinal wall image on a screen; and display an opening portion image simulating an opening portion existing in a duodenal papilla, in the duodenal papilla region in the intestinal wall image displayed on the screen.

### CROSS-REFERENCE TO RELATED APPLICATIONS

This application is a continuation application of International Application No. PCT/JP2023/036267, filed Oct. 4, 2023, the disclosure of which is incorporated herein by reference in its entirety. Further, this application claims priority from Japanese Patent Application No. 2022-177611, filed Nov. 4, 2022, the disclosure of which is incorporated herein by reference in its entirety.

### BACKGROUND

### 1. Technical Field

The technology of the present disclosure relates to a medical support device, an endoscope, a medical support method, and a program.

### 2. Related Art

JP2020-62218A discloses a learning device including an acquisition unit that acquires a plurality of pieces of information in which an image of a Vater's papilla of a duodenum of a bile duct is associated with information indicating a cannulation method being a method of inserting a catheter into the bile duct; a learning unit that performs machine learning while the information indicating the cannulation method is used as training data based on the image of the Vater's papilla of the duodenum of the bile duct; and a storage unit that stores a result of the machine learning of the learning unit in association with the information indicating the cannulation method.

### SUMMARY

One embodiment according to the technology of the present disclosure provides a medical support device, an endoscope, a medical support method, and a program capable of causing information that is used for a treatment on a duodenal papilla to be visually recognized.

A first aspect according to the technology of the present disclosure is a medical support device including a processor. The processor is configured to detect a duodenal papilla region by executing image recognition processing on an intestinal wall image obtained by a camera provided in an endoscopic scope imaging an intestinal wall of a duodenum; display the intestinal wall image on a screen; and display an opening portion image simulating an opening portion existing in a duodenal papilla, in the duodenal papilla region in the intestinal wall image displayed on the screen.

A second aspect according to the technology of the present disclosure is the medical support device according to the first aspect, in which the opening portion image includes a first pattern image selected in accordance with a given first instruction from a plurality of first pattern images expressing different first geometric features of the opening portion in the duodenal papilla.

A third aspect according to the technology of the present disclosure is the medical support device according to the second aspect, in which the plurality of first pattern images are displayed one by one as the opening portion image on the screen, and the first pattern image displayed as the opening portion image on the screen is switched in response to the first instruction.

A fourth aspect according to the technology of the present disclosure is the medical support device according to the second aspect or the third aspect, in which the first geometric feature is a position and/or a size of the opening portion in the duodenal papilla.

A fifth aspect according to the technology of the present disclosure is the medical support device according to any one of the first aspect to the fourth aspect, in which the opening portion image is an image created based on a first reference image obtained by one or more modalities and/or first information obtained from a medical finding.

A sixth aspect according to the technology of the present disclosure is the medical support device according to any one of the first aspect to the fifth aspect, in which the opening portion image includes a map indicating a distribution of a probability that the opening portion exists in the duodenal papilla.

A seventh aspect according to the technology of the present disclosure is the medical support device according the sixth aspect, in which the image recognition processing is AI-based image recognition processing, and the distribution of the probability is obtained by the image recognition processing being executed.

An eighth aspect according to the technology of the present disclosure is the medical support device according to any one of the first aspect to the seventh aspect, in which a size of the opening portion image changes in accordance with a size of the duodenal papilla region in the screen.

A ninth aspect according to the technology of the present disclosure is the medical support device according to any one of the first aspect to the eighth aspect, in which the opening portion consists of one or more openings.

A tenth aspect according to the technology of the present disclosure is the medical support device according to any one of the first aspect to the ninth aspect, in which the processor displays a duct path image indicating a path of one or more ducts that are a bile duct and/or a pancreatic duct in accordance with the duodenal papilla region, in the intestinal wall image displayed on the screen.

An eleventh aspect according to the technology of the present disclosure is the medical support device according to the tenth aspect, in which the duct path image includes a second pattern image selected in accordance with a given second instruction from a plurality of second pattern images expressing different second geometric features of the duct in the intestinal wall.

A twelfth aspect according to the technology of the present disclosure is the medical support device according to the eleventh aspect, in which the plurality of second pattern images are displayed one by one as the duct path image on the screen, and the second pattern image displayed as the duct path image on the screen is switched in response to the second instruction.

A thirteenth aspect according to the technology of the present disclosure is the medical support device according to the eleventh aspect or the twelfth aspect, in which the second geometric feature is a position and/or a size of the path in the intestinal wall.

A fourteenth aspect according to the technology of the present disclosure is the medical support device according to any one of the tenth aspect to the thirteenth aspect, in which the duct path image is an image created based on a second reference image obtained by one or more modalities and/or second information obtained from a medical finding.

A fifteenth aspect according to the technology of the present disclosure is the medical support device according to any one of the tenth aspect to the fourteenth aspect, in which an image in which the duct path image is included in the intestinal wall image is stored in an external device and/or a medical record.

A sixteenth aspect according to the technology of the present disclosure is the medical support device according to any one of the first aspect to the fifteenth aspect, in which an image in which the opening portion image is included in the duodenal papilla region is stored in an external device and/or a medical record.

A seventeenth aspect according to the technology of the present disclosure is a medical support device including a processor. The processor is configured to detect a duodenal papilla region by executing image recognition processing on an intestinal wall image obtained by a camera provided in an endoscopic scope imaging an intestinal wall of a duodenum; display the intestinal wall image on a screen; and display a duct path image indicating a path of one or more ducts that are a bile duct and/or a pancreatic duct in accordance with the duodenal papilla region, in the intestinal wall image displayed on the screen.

An eighteenth aspect according to the technology of the present disclosure is an endoscope including the medical support device according to any one of the first aspect to the seventeenth aspect; and the endoscopic scope.

A nineteenth aspect according to the technology of the present disclosure is a medical support method including detecting a duodenal papilla region by executing image recognition processing on an intestinal wall image obtained by a camera provided in an endoscopic scope imaging an intestinal wall of a duodenum; displaying the intestinal wall image on a screen; and displaying an opening portion image simulating an opening portion existing in a duodenal papilla, in the duodenal papilla region in the intestinal wall image displayed on the screen.

A twentieth aspect according to the technology of the present disclosure is a medical support method including detecting a duodenal papilla region by executing image recognition processing on an intestinal wall image obtained by a camera provided in an endoscopic scope imaging an intestinal wall of a duodenum; displaying the intestinal wall image on a screen; and displaying a duct path image indicating a path of one or more ducts that are a bile duct and/or a pancreatic duct in accordance with the duodenal papilla region, in the intestinal wall image displayed on the screen.

A twenty-first aspect according to the technology of the present disclosure is a program for causing a computer to execute processing including detecting a duodenal papilla region by executing image recognition processing on an intestinal wall image obtained by a camera provided in an endoscopic scope imaging an intestinal wall of a duodenum; displaying the intestinal wall image on a screen; and displaying an opening portion image simulating an opening portion existing in a duodenal papilla, in the duodenal papilla region in the intestinal wall image displayed on the screen.

A twenty-second aspect according to the technology of the present disclosure is a program for causing a computer to execute processing including detecting a duodenal papilla region by executing image recognition processing on an intestinal wall image obtained by a camera provided in an endoscopic scope imaging an intestinal wall of a duodenum; displaying the intestinal wall image on a screen; and displaying a duct path image indicating a path of one or more ducts that are a bile duct and/or a pancreatic duct in accordance with the duodenal papilla region, in the intestinal wall image displayed on the screen.

### BRIEF DESCRIPTION OF THE DRAWINGS

Exemplary embodiments according to the technique of the present disclosure will be described in detail based on the following figures, wherein:

FIG. 1 is a conceptual view illustrating an example of an aspect in which a duodenoscope system is used;

FIG. 2 is a conceptual view illustrating an example of the overall configuration of the duodenoscope system;

FIG. 3 is a block diagram illustrating an example of a hardware configuration of an electrical system of the duodenoscope system;

FIG. 4 is a conceptual view illustrating an example of an aspect in which a duodenoscope is used;

FIG. 5 is a block diagram illustrating an example of a hardware configuration of an electrical system of an image processing device;

FIG. 6 is a conceptual diagram illustrating an example of the correlation among an endoscopic scope, a NVM, an image acquisition unit, an image recognition unit, and an image adjustment unit;

FIG. 7 is a block diagram illustrating an example of main functions of an opening portion image generation device;

FIG. 8 is a conceptual diagram illustrating an example of the correlation among a display device, the image acquisition unit, the image recognition unit, the image adjustment unit, and a display control unit;

FIG. 9 is a conceptual diagram illustrating an example of an aspect in which an opening portion image is switched;

FIG. 10 is a flowchart presenting an example of the flow of medical support processing;

FIG. 11 is a conceptual diagram illustrating an example of the correlation among the endoscopic scope, the image acquisition unit, the image recognition unit, and the image adjustment unit;

FIG. 12 is a conceptual diagram illustrating an example of the correlation among the display device, the image acquisition unit, the image recognition unit, the image adjustment unit, and the display control unit;

FIG. 13 is a conceptual diagram illustrating an example of the correlation among the endoscopic scope, the NVM, the image acquisition unit, the image recognition unit, and the image adjustment unit;

FIG. 14 is a block diagram illustrating an example of main functions of a duct path image generation device;

FIG. 15 is a conceptual diagram illustrating an example of the correlation among the display device, the image acquisition unit, the image recognition unit, the image adjustment unit, and the display control unit;

FIG. 16 is a conceptual diagram illustrating an example of an aspect in which a duct path image is switched;

FIG. 17 is a flowchart presenting an example of the flow of medical support processing;

FIG. 18 is a conceptual diagram illustrating an example of the correlation among the endoscopic scope, the NVM, the image acquisition unit, the image recognition unit, and the image adjustment unit;

FIG. 19 is a conceptual diagram illustrating an example of the correlation among the display device, the image acquisition unit, the image recognition unit, the image adjustment unit, and the display control unit;

FIG. 20 is a conceptual diagram illustrating an example of an aspect in which the opening portion image and the duct path image are switched; and

FIG. 21 is a conceptual diagram illustrating an example of an aspect in which the opening portion image and the duct path image generated by the duodenoscope system are stored in an electronic medical record server.

### DETAILED DESCRIPTION

Hereinafter, an example of an embodiment of a medical support device, an endoscope, a medical support method, and a program according to the technology of the present disclosure will be described with reference to the accompanying drawings.

First, terms used in the following description will be described.

CPU is an abbreviation of “Central Processing Unit”. GPU is an abbreviation of “Graphics Processing Unit”. RAM is an abbreviation of “Random Access Memory”. NVM is an abbreviation of “Non-volatile memory”. EEPROM is an abbreviation of “Electrically Erasable Programmable Read-Only Memory”. ASIC is an abbreviation of “Application Specific Integrated Circuit”. PLD is an abbreviation of “Programmable Logic Device”. FPGA is an abbreviation of “Field-Programmable Gate Array”. SoC is an abbreviation of “System-on-a-chip”. SSD is an abbreviation of “Solid State Drive”. USB is an abbreviation of “Universal Serial Bus”. HDD is an abbreviation of “Hard Disk Drive”. EL is an abbreviation of “Electro-Luminescence”. CMOS is an abbreviation of “Complementary Metal Oxide Semiconductor”. CCD is an abbreviation of “Charge Coupled Device”. AI is an abbreviation of “Artificial Intelligence”. BLI is an abbreviation of “Blue Light Imaging”. LCI is an abbreviation of “Linked Color Imaging”. I/F is an abbreviation of “Interface”. FIFO is an abbreviation of “First In First Out”. ERCP is an abbreviation of “Endoscopic Retrograde Cholangio-Pancreatography”. CT is an abbreviation of “Computed Tomography”. MRI is an abbreviation of “Magnetic Resonance Imaging”.

### First Embodiment

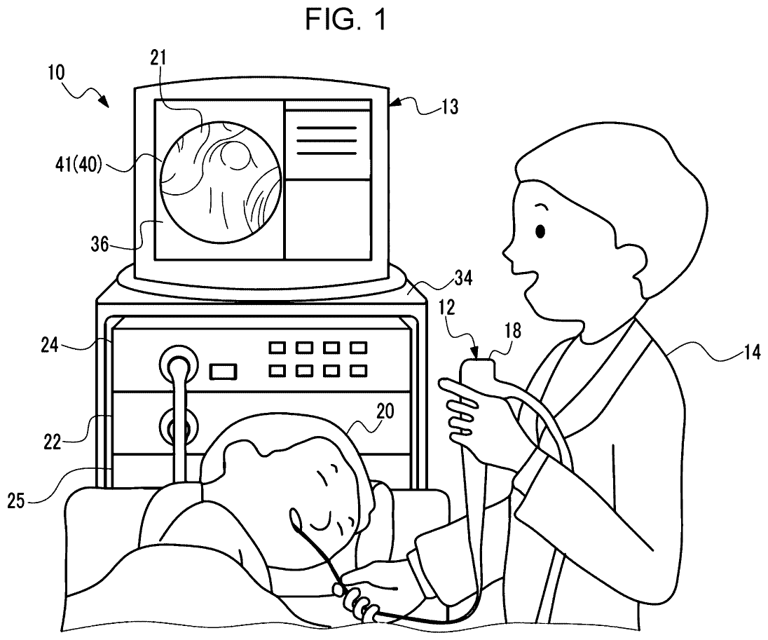

In one example, as illustrated in FIG. 1, a duodenoscope system 10 includes a duodenoscope 12 and a display device 13. The duodenoscope 12 is used by a physician 14 in endoscope examinations. The duodenoscope 12 is communicably connected to a communication device (not illustrated), and information obtained by the duodenoscope 12 is transmitted to the communication device. The communication device receives the information transmitted from the duodenoscope 12, and executes processing using the received information (for example, processing of recording the information in an electronic medical record or the like).

The duodenoscope 12 includes an endoscopic scope 18. The duodenoscope 12 is a device for performing medical diagnosis and treatment on an observation target 21 (for example, an upper gastrointestinal tract) included in the body of a subject 20 (for example, a patient) using the endoscopic scope 18. The observation target 21 is a target to be observed by the physician 14. The endoscopic scope 18 is inserted into the body of the subject 20. The duodenoscope 12 causes the endoscopic scope 18 inserted into the body of the subject 20 to image the observation target 21 in the body of the subject 20, and performs various medical treatments on the observation target 21 as necessary. The duodenoscope 12 is an example of an “endoscope” according to the technology of the present disclosure.

The duodenoscope 12 images the inside of the body of the subject 20 to acquire and output an image indicating the state of the inside of the body. In the present embodiment, the duodenoscope 12 is an endoscope having an optical imaging function of imaging reflected light obtained through irradiation with light in the body and reflection of the light from the observation target 21.

The duodenoscope 12 includes a control device 22, a light source device 24, and an image processing device 25. The control device 22 and the light source device 24 are installed in a wagon 34. In the wagon 34, a plurality of shelfs are provided along the vertical direction, and the image processing device 25, the control device 22, and the light source device 24 are installed from the shelf on the lower side to the shelf on the upper side. Also, the display device 13 is installed on the top shelf of the wagon 34.

The control device 22 is a device that controls the entirety of the duodenoscope 12. Also, the image processing device 25 is a device that performs image processing on an image captured by the duodenoscope 12 under the control of the control device 22.

The display device 13 displays various kinds of information including an image (for example, an image on which the image processing has been performed by the image processing device 25). An example of the display device 13 is a liquid crystal display or an EL display. A tablet terminal with a display may be used instead of the display device 13 or together with the display device 13.

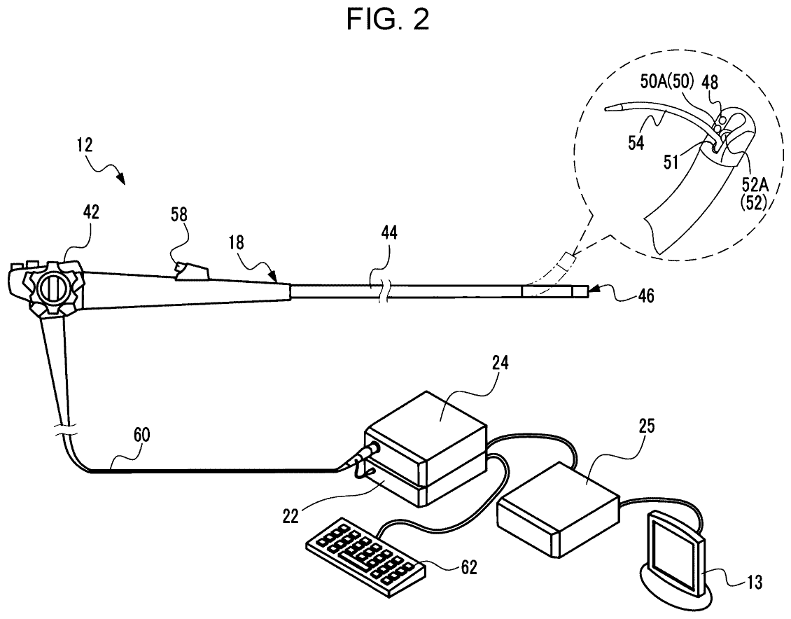

In the example illustrated in FIG. 1, a screen 36 is provided at the display device 13. An endoscopic image 40 obtained by the duodenoscope 12 is displayed on the screen 36. The observation target 21 is shown in the endoscopic image 40. The endoscopic image 40 is an image obtained by a camera 48 (see FIG. 2) provided in the endoscopic scope 18 imaging the observation target 21 in the body of the subject 20. An example of the observation target 21 is the intestinal wall of the duodenum. Hereinafter, for the convenience of description, an intestinal wall image 41 that is the endoscopic image 40 in which the intestinal wall of the duodenum is imaged as the observation target 21 will be described as an example. Note that the duodenum is merely an example, and the observation target 21 may be any region that can be imaged by the duodenoscope 12. An example of the region that can be imaged by the duodenoscope 12 is the esophagus, the stomach, or the like. The intestinal wall image 41 is an example of an “intestinal wall image” according to the technology of the present disclosure.

A moving image constituted by including a plurality of frames of intestinal wall images 41 is displayed on the screen 36. That is, the plurality of frames of the intestinal wall images 41 are displayed on the screen 36 at a predetermined frame rate (for example, several tens of frames/second).

In one example, as illustrated in FIG. 2, the duodenoscope 12 includes an operation section 42 and an insertion section 44. The insertion section 44 is partially bent when the operation section 42 is operated. The insertion section 44 is inserted while being bent along the shape of the observation target 21 (for example, the shape of the duodenum) in accordance with the operation of the operation section 42 by the physician 14.

A tip portion 46 of the insertion section 44 is provided with the camera 48, an illumination device 50, a treatment opening 51, and a rising mechanism 52. The camera 48 and the illumination device 50 are provided in a side surface of the tip portion 46. That is, the duodenoscope 12 is a side-view scope. Accordingly, it is easy to observe the intestinal wall of the duodenum.

The camera 48 is a device that acquires the intestinal wall image 41 as a medical image by imaging the inside of the body of the subject 20. An example of the camera 48 is a CMOS camera. However, this is merely an example, and another type of camera such as a CCD camera may be used. The camera 48 is an example of a “camera” according to the technology of the present disclosure.

The illumination device 50 has an illumination window 50A. The illumination device 50 emits light via the illumination window 50A. Examples of the type of light to be emitted from the illumination device 50 include visible light (for example, white light or the like) and invisible light (for example, near-infrared light or the like). Additionally or alternatively, the illumination device 50 emits special light via the illumination window 50A. Examples of the special light include light for BLI and/or light for LCI. The camera 48 images the inside of the body of the subject 20 by an optical method in a state in which light is emitted by the illumination device 50 in the body of the subject 20.

The treatment opening 51 is used as a treatment tool protrusion port for allowing a treatment tool 54 to protrude from the tip portion 46, a suction port for sucking blood, body waste, and the like, and a delivery port for delivering a fluid.

The treatment tool 54 protrudes from the treatment opening 51 in accordance with the operation of the physician 14. The treatment tool 54 is inserted into the insertion section 44 from a treatment tool insertion port 58. The treatment tool 54 passes through the inside of the insertion section 44 via the treatment tool insertion port 58 and protrudes into the body of the subject 20 from the treatment opening 51. In the example illustrated in FIG. 2, as the treatment tool 54, a cannula protrudes from the treatment opening 51. The cannula is merely an example of the treatment tool 54, and another example of the treatment tool 54 is a papillotomy knife, a snare, or the like.

The rising mechanism 52 changes the protruding direction of the treatment tool 54 protruding from the treatment opening 51. The rising mechanism 52 includes a guide 52A, and when the guide 52A rises with respect to the protruding direction of the treatment tool 54, the protruding direction of the treatment tool 54 changes along the guide 52A. Accordingly, it is easy for the treatment tool 54 to protrude toward the intestinal wall. In the example illustrated in FIG. 2, the protruding direction of the treatment tool 54 is changed to a direction orthogonal to the advancing direction of the tip portion 46 by the rising mechanism 52. The rising mechanism 52 is operated by the physician 14 via the operation section 42. Accordingly, the degree of change in the protruding direction of the treatment tool 54 is adjusted.

The endoscopic scope 18 is connected to the control device 22 and the light source device 24 via a universal cord 60. The display device 13 and a reception device 62 are connected to the control device 22. The reception device 62 receives an instruction from a user (for example, the physician 14) and outputs the received instruction as an electric signal. In the example illustrated in FIG. 2, an example of the reception device 62 is a keyboard. However, this is merely an example, and the reception device 62 may be a mouse, a touch panel, a foot switch, a microphone, and/or the like.

The control device 22 controls the entirety of the duodenoscope 12. For example, the control device 22 controls the light source device 24, and transmits and receives various signals to and from the camera 48. The light source device 24 emits light under the control of the control device 22 and supplies the light to the illumination device 50. A light guide is built in the illumination device 50, and the light supplied from the light source device 24 is emitted from the illumination windows 50A via the light guide. The control device 22 causes the camera 48 to perform imaging, acquires an intestinal wall image 41 (see FIG. 1) from the camera 48, and outputs the intestinal wall image 41 to a predetermined output destination (for example, the image processing device 25).

The image processing device 25 is communicably connected to the control device 22, and the image processing device 25 performs image processing on the intestinal wall image 41 output from the control device 22. Details of the image processing in the image processing device 25 will be described later. The image processing device 25 outputs the intestinal wall image 41 subjected to the image processing to a predetermined output destination (for example, the display device 13). Here, the embodiment example in which the intestinal wall image 41 output from the control device 22 is output to the display device 13 via the image processing device 25 has been described, but this is merely an example. An aspect in which the control device 22 and the display device 13 are connected to each other, and the intestinal wall image 41 subjected to the image processing by the image processing device 25 is displayed on the display device 13 via the control device 22 may be employed.

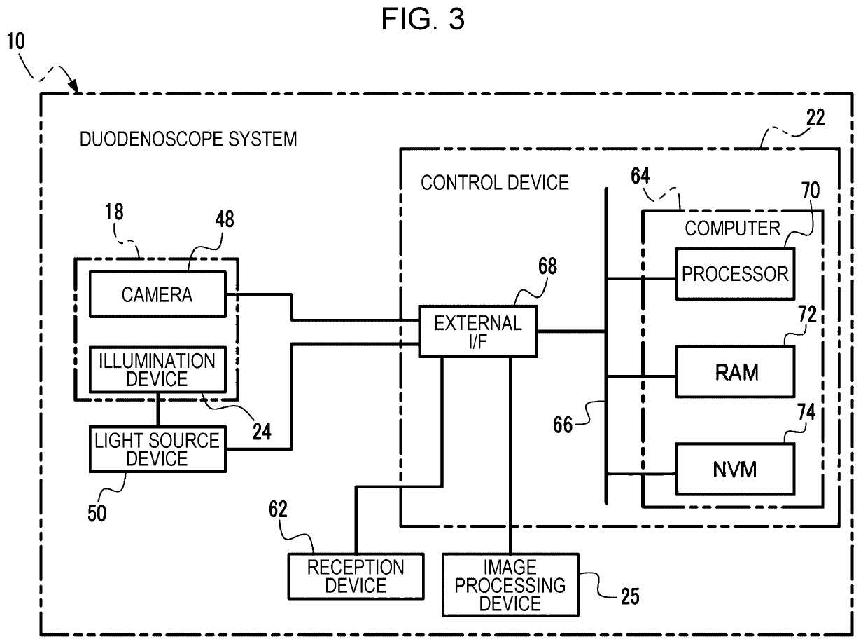

In one example, as illustrated in FIG. 3, the control device 22 includes a computer 64, a bus 66, and an external I/F 68. The computer 64 includes a processor 70, a RAM 72, and a NVM 74. The processor 70, the RAM 72, the NVM 74, and the external I/F 68 are connected to the bus 66.

For example, the processor 70 has a CPU and a GPU, and controls the entirety of the control device 22. The GPU operates under the control of the CPU, and is in charge of execution of various types of processing of a graphic system, arithmetic operation using a neural network, and the like. Note that the processor 70 may be one or more CPUs in which the GPU function is integrated, or may be one or more CPUs in which the GPU function is not integrated.

The RAM 72 is a memory in which information is temporarily stored, and is used as a work memory by the processor 70. The NVM 74 is a non-volatile storage device that stores various programs, various parameters, and the like. An example of the NVM 74 is a flash memory (for example, an EEPROM and/or a SSD). Note that the flash memory is merely an example, and may be another non-volatile storage device such as a HDD, or may be a combination of two or more types of non-volatile storage devices.

The external I/F 68 is in charge of transmission and reception of various kinds of information between devices existing outside the control device 22 (hereinafter, also referred to as “external devices”) and the processor 70. An example of the external I/F 68 is a USB interface.

The camera 48 as one of the external devices is connected to the external I/F 68, and the external I/F 68 is in charge of transmission and reception of various kinds of information between the camera 48 provided in the endoscopic scope 18 and the processor 70. The processor 70 controls the camera 48 via the external I/F 68. Also, the processor 70 acquires an intestinal wall image 41 (refer to FIG. 1) obtained by the camera 48 provided in the endoscopic scope 18 imaging the inside of the body of the subject 20, via the external I/F 68.

The light source device 24 as one of the external devices is connected to the external I/F 68, and the external I/F 68 is in charge of transmission and reception of various kinds of information between the light source device 24 and the processor 70. The light source device 24 supplies light to the illumination device 50 under the control of the processor 70. The illumination device 50 emits the light supplied from the light source device 24.

The reception device 62 as one of the external devices is connected to the external I/F 68, and the processor 70 acquires an instruction received by the reception device 62 via the external I/F 68 and executes processing in accordance with the acquired instruction.

The image processing device 25 as one of the external devices is connected to the external I/F 68, and the processor 70 outputs the intestinal wall image 41 to the image processing device 25 via the external I/F 68.

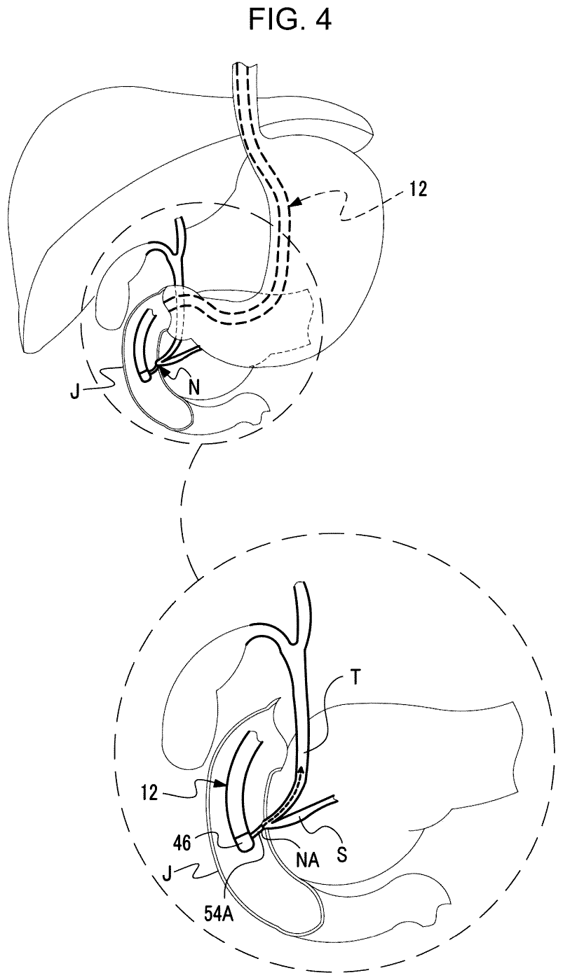

Meanwhile, in some cases, a treatment called an Endoscopic Retrograde Cholangio-Pancreatography (ERCP) examination is performed among treatments on a duodenum using an endoscope. In one example, as illustrated in FIG. 4, in the ERCP examination, for example, the duodenoscope 12 is inserted to a duodenum J via the esophagus and the stomach first. In this case, the insertion state of the duodenoscope 12 may be checked using X-ray imaging. Then, the tip portion 46 of the duodenoscope 12 reaches the vicinity of a duodenal papilla N (hereinafter, also simply referred to as a “papilla N”) existing in the intestinal wall of the duodenum J.

In the ERCP examination, for example, a cannula 54A is inserted from the papilla N. Here, the papilla N is an area bulging from the intestinal wall of the duodenum J, and openings of end portions of a bile duct T (for example, the common bile duct, the intrahepatic bile duct, the cystic duct) and a pancreatic duct S exist in a papilla bulge NA of the papilla N. X-ray imaging is performed in a state in which a contrast medium is injected into the bile duct T, the pancreatic duct S, and the like from the opening of the papilla N via the cannula 54A. In the ERCP examination, it is important to perform a treatment after grasping the state of the papilla N (for example, the position, the size, and/or the type of the papilla N) or the states of the bile duct T and the pancreatic duct S (for example, the running paths of the ducts). This is because, when the cannula 54A is inserted, the state of the papilla N affects the success or failure of the insertion, and the states of the bile duct T and the pancreatic duct S affect the success or failure of the intubation after the insertion. However, for example, since the physician 14 operates the duodenoscope 12, it is difficult to always grasp the state of the papilla N or the states of the bile duct T and the pancreatic duct S.

In view of such circumstances, in the present embodiment, medical support processing is performed by a processor 82 of the image processing device 25 in order to allow the user to visually recognize information that is used for a treatment on the papilla.

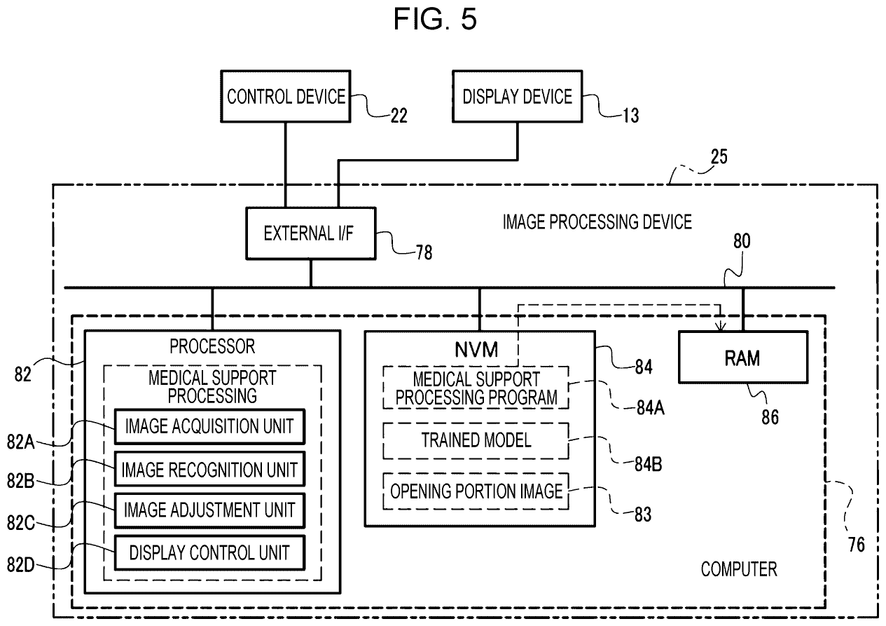

In one example, as illustrated in FIG. 5, the image processing device 25 includes a computer 76, an external I/F 78, and a bus 80. The computer 76 includes a processor 82, a NVM 84, and a RAM 86. The processor 82, the NVM 84, the RAM 86, and the external I/F 78 are connected to the bus 80. The computer 76 is an example of a “medical support device” and a “computer” according to the technology of the present disclosure. The processor 82 is an example of a “processor” according to the technology of the present disclosure.

Note that the hardware configuration of the computer 76 (that is, the processor 82, the NVM 84, and the RAM 86) is basically the same as the hardware configuration of the computer 64 illustrated in FIG. 3, and hence the description relating to the hardware configuration of the computer 76 will be omitted here. Also, the role of the external I/F 78 in the image processing device 25 for transmitting and receiving information to and from the outside is basically the same as the role of the external I/F 68 in the control device 22 illustrated in FIG. 3, and hence the description thereof will be omitted here.

A medical support processing program 84A is stored in the NVM 84. The medical support processing program 84A is an example of a “program” according to the technology of the present disclosure. The processor 82 reads out the medical support processing program 84A from the NVM 84, and executes the read-out medical support processing program 84A on the RAM 86. The medical support processing is implemented by the processor 82 operating as an image acquisition unit 82A, an image recognition unit 82B, an image adjustment unit 82C, and a display control unit 82D in accordance with the medical support processing program 84A executed on the RAM 86.

In the NVM 84, a trained model 84B is stored. In the present embodiment, the image recognition unit 82B performs AI-based image recognition processing as image recognition processing for object detection. The trained model 84B has been optimized by performing machine learning on a neural network in advance.

An opening portion image 83 is stored in the NVM 84. The opening portion image 83 is an image created in advance, and is an image simulating an opening portion existing in the papilla N. The opening portion image 83 is an example of an “opening portion image” according to the technology of the present disclosure. Details of the opening portion image 83 will be described later.

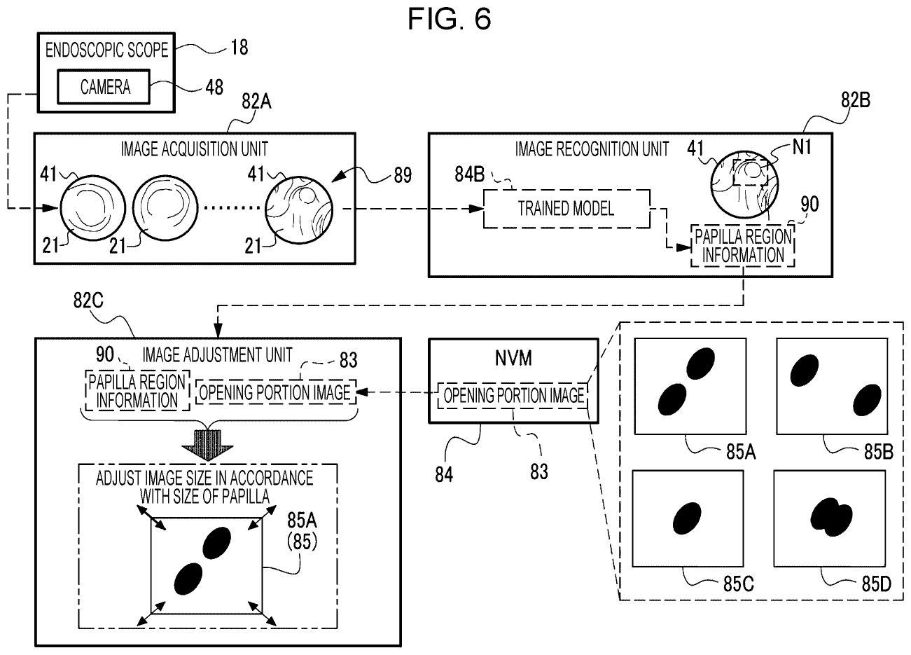

In one example, as illustrated in FIG. 6, the image acquisition unit 82A acquires, from the camera 48, an intestinal wall image 41 generated by being captured by the camera 48 provided in the endoscopic scope 18 in accordance with an imaging frame rate (for example, several tens of frames/second) on a frame-by-frame basis.

The image acquisition unit 82A holds a time-series image group 89. The time-series image group 89 is a plurality of time-series intestinal wall images 41 in which the observation target 21 is shown. The time-series image group 89 includes, for example, a constant number of frames (for example, a predetermined number of frames in a range of several tens to several hundreds of frames) of intestinal wall images 41. The image acquisition unit 82A updates the time-series image group 89 by a FIFO method every time the image acquisition unit 82A acquires an intestinal wall image 41 from the camera 48.

Here, the embodiment example in which the time-series image group 89 is held and updated by the image acquisition unit 82A has been described, but this is merely an example. For example, the time-series image group 89 may be held and updated in a memory, such as the RAM 86, connected to the processor 82.

The image recognition unit 82B performs image recognition processing on the time-series image group 89 using the trained model 84B. By performing the image recognition processing, the papilla N included in the observation target 21 is detected. In other words, by performing the image recognition processing, a duodenal papilla region N1 (hereinafter, also simply referred to as a “papilla region N1”) that is a region indicating the papilla N included in the intestinal wall image 41 is detected. In the present embodiment, the detection of the papilla region N1 represents processing of specifying the papilla region N1 and storing papilla region information 90 and the intestinal wall image 41 in the memory in a state of being associated with each other. Here, the papilla region information 90 includes information (for example, coordinates and a range in the image) for allowing the papilla region N1 to be specified in the intestinal wall image 41 in which the papilla N is shown. The papilla region N1 is an example of a “duodenal papilla region” according to the technology of the present disclosure.

The trained model 84B is obtained by optimizing a neural network by performing machine learning on the neural network using training data. The training data is a plurality of data (that is, data of a plurality of frames) in which example data and correct answer data are associated with each other. The example data is, for example, an image (for example, an image corresponding to the intestinal wall image 41) obtained by imaging an area (for example, the inner wall of the duodenum) that can be a target of the ERCP examination. The correct answer data is an annotation corresponding to the example data. An example of the correct answer data is an annotation capable of specifying the papilla region N1.

Here, the embodiment example in which only one trained model 84B is used by the image recognition unit 82B has been described, but this is merely an example. For example, a trained model 84B selected from a plurality of trained models 84B may be used by the image recognition unit 82B. In this case, each trained model 84B may be created by performing machine learning specialized for each procedure of the ERCP examination (for example, the position or the like of the duodenoscope 12 with respect to the papilla N), and the trained model 84B corresponding to the procedure of the currently performed ERCP examination may be selected and used by the image recognition unit 82B.

The image recognition unit 82B acquires the time-series image group 89 from the image acquisition unit 82A, and inputs the acquired time-series image group 89 to the trained model 84B. Accordingly, the trained model 84B outputs papilla region information 90 corresponding to the input time-series image group 89. The image recognition unit 82B acquires the papilla region information 90 output from the trained model 84B. The papilla region N1 may be detected by a bounding box used in the image recognition processing, or may be detected by segmentation (for example, semantic segmentation).

The image adjustment unit 82C acquires the papilla region information 90 from the image recognition unit 82B. Also, the image adjustment unit 82C acquires an opening portion image 83 from the NVM 84. The opening portion image 83 includes a plurality of opening portion pattern images 85A to 85D. In the following description, when the plurality of opening portion pattern images 85A to 85D are not distinguished from each other, they are also simply referred to as “opening portion pattern images 85”. The plurality of opening portion pattern images 85 are images expressing different geometric features of the opening portion. Here, the geometric feature of the opening portion represents the position and/or the size of the opening portion in the papilla N. That is, the plurality of opening portion pattern images 85 are different from each other in the position and/or the size of the opening portion. The opening portion pattern image 85 is an example of a “first pattern image” according to the technology of the present disclosure.

The opening portion indicated by the opening portion image 83 consists of one or more openings. The opening portion pattern image 85 is generated, for example, by simulating an opening portion corresponding to the classification (for example, a separate opening type, an onion type, a nodule type, a villous type, or the like) of the papilla N. For example, in the case of the separate opening type, the opening portion pattern image 85 is an opening portion pattern image 85 simulating an opening portion including the opening of the bile duct T and the opening of the pancreatic duct S, and two openings are presented in the opening portion pattern image 85. Here, the example in which the four opening portion pattern images 85A to 85D are included in the opening portion image 83 has been described, but this is merely an example, and the number of images included in the opening portion image 83 may be two or three, or may be five or more.

The image adjustment unit 82C adjusts the size of the opening portion image 83 in accordance with the size of the papilla region N1 indicated by the papilla region information 90. The image adjustment unit 82C adjusts the size of the opening portion image 83 using, for example, an adjustment table (not illustrated). The adjustment table is a table in which the size of the papilla region N1 is set as an input value and the size of the opening portion image 83 is set as an output value. By enlarging or reducing the opening portion image 83, the size of the opening portion image 83 is adjusted. Here, the embodiment example in which the size of the opening portion image 83 is adjusted using the adjustment table has been described, but this is merely an example. For example, the size of the opening portion image 83 may be adjusted using an adjustment arithmetic expression. The adjustment arithmetic expression is an arithmetic expression in which the size of the papilla region N1 is an independent variable and the size of the opening portion image 83 is a dependent variable.

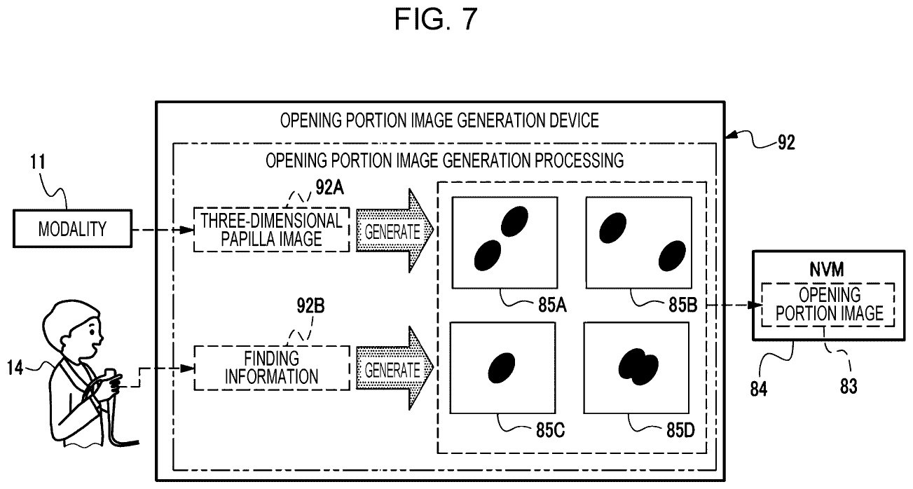

In one example, as illustrated in FIG. 7, the opening portion image 83 is generated by an opening portion image generation device 92. The opening portion image generation device 92 is an external device connectable to the image processing device 25. The hardware configuration (for example, a processor, a NVM, a RAM, and the like) of the opening portion image generation device 92 is basically the same as the hardware configuration of the control device 22 illustrated in FIG. 3, and hence the description relating to the hardware configuration of the opening portion image generation device 92 will be omitted here.

In the opening portion image generation device 92, opening portion image generation processing is executed. In the opening portion image generation processing, a three-dimensional papilla image 92A is generated based on volume data obtained by a modality 11 (for example, a CT apparatus or a MRI apparatus). Further, rendering by viewing the three-dimensional papilla image 92A from a predetermined viewpoint (for example, a viewpoint directly facing the papilla) is performed, thereby generating an opening portion pattern image 85. The three-dimensional papilla image 92A is an example of a “first reference image” according to the technology of the present disclosure.

Also, in the opening portion image generation processing, opening portion pattern image 85 is generated based on finding information 92B input by the physician 14 via the reception device 62. Here, the finding information 92B is information indicating the position, the shape, and/or the size of the opening portion indicated by a medical finding. The finding information 92B is an example of “first information” according to the technology of the present disclosure. To be specific, for example, the physician 14 inputs the finding information 92B by designating the position and the size of the opening portion using a keyboard as the reception device 62. In another example, finding information 92B is generated based on a statistical value (for example, the mode) of position coordinates of a region diagnosed as an opening portion in a past examination. The opening portion image generation device 92 outputs a plurality of opening portion pattern images 85 generated in the opening portion image generation processing to the NVM 84 of the image processing device 25.

Here, the embodiment example in which the opening portion image 83 is generated in the opening portion image generation device 92 has been described, but the technology of the present disclosure is not limited thereto. For example, an aspect in which the image processing device 25 has a function equivalent to that of the opening portion image generation device 92, and the opening portion image 83 is generated in the image processing device 25 may be employed.

Also, here, the embodiment example in which the opening portion image 83 is generated from the three-dimensional papilla image 92A and the finding information 92B has been described, but the technology of the present disclosure is not limited thereto. For example, the opening portion image 83 may be generated from any one of the three-dimensional papilla image 92A and the finding information 92B.

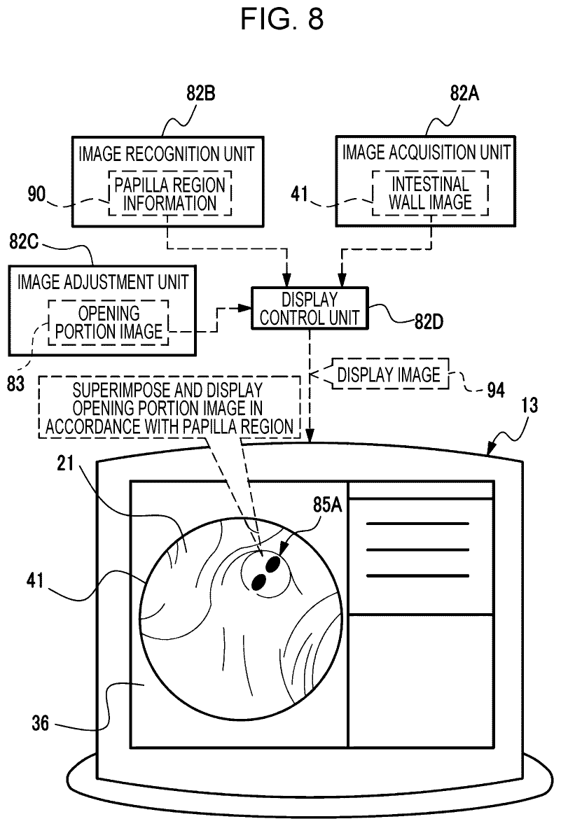

In one example, as illustrated in FIG. 8, the display control unit 82D acquires an intestinal wall image 41 from the image acquisition unit 82A. Also, the display control unit 82D acquires papilla region information 90 from the image recognition unit 82B. Further, the display control unit 82D acquires an opening portion image 83 from the image adjustment unit 82C. Here, the image size of the opening portion image 83 is adjusted by the image adjustment unit 82C in accordance with the size of the papilla region N1.

The display control unit 82D superimposes and displays the opening portion image 83 in the papilla region N1 in the intestinal wall image 41. To be specific, the display control unit 82D displays the opening portion image 83 whose image size has been adjusted at the position of the papilla region N1 indicated by the papilla region information 90 in the intestinal wall image 41. Accordingly, the opening portion indicated by the opening portion image 83 is displayed in the papilla region N1 in the intestinal wall image 41. Further, the display control unit 82D generates a display image 94 including the intestinal wall image 41 on which the opening portion image 83 has been superimposed and outputs the display image 94 to the display device 13. To be specific, the display control unit 82D causes the display device 13 to display the screen 36 by performing Graphical User Interface (GUI) control for displaying the display image 94. The screen 36 is an example of a “screen” according to the technology of the present disclosure. In the example illustrated in FIG. 8, the opening portion pattern image 85A is superimposed and displayed on the intestinal wall image 41. For example, the physician 14 visually recognizes the opening portion pattern image 85 A displayed on the screen 36 and uses the opening portion pattern image 85A as a guide for inserting a cannula into the papilla N. Note that the opening portion pattern image 85 to be displayed first may be determined in advance, or may be designated by the user.

Also, when the intestinal wall image 41 is displayed in an enlarged or reduced manner by the user's operation, the opening portion image 83 is also enlarged or reduced in accordance with the enlargement or the reduction of the intestinal wall image 41. In this case, the image adjustment unit 82C adjusts the size of the opening portion image 83 in accordance with the size of the intestinal wall image 41. Then, the display control unit 82D superimposes and displays the opening portion image 83 whose size has been adjusted on the intestinal wall image 41.

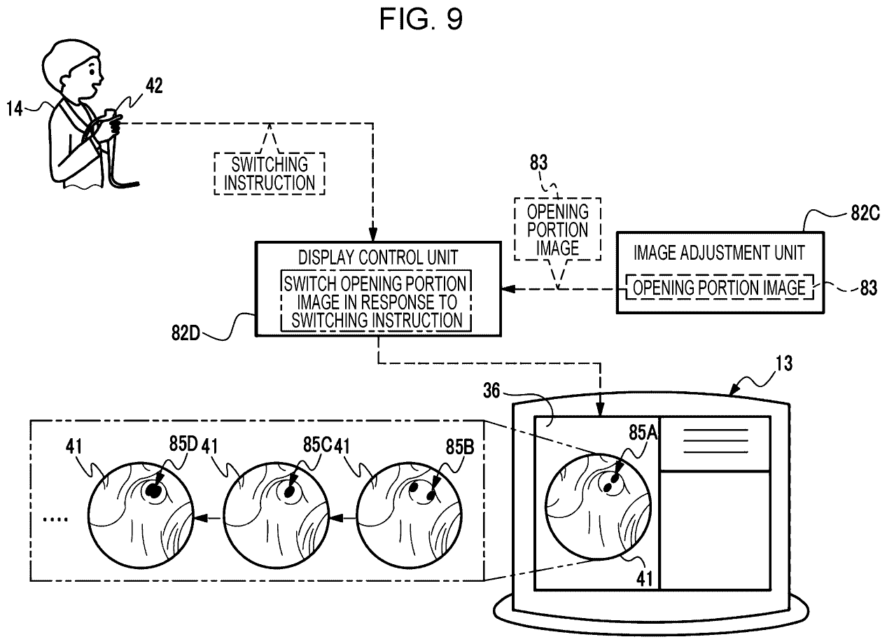

In one example, as illustrated in FIG. 9, the display control unit 82D performs processing of switching in response to a switching instruction from the physician 14. The physician 14 inputs the switching instruction of the opening portion image 83, for example, via the operation section 42 (for example, an operation knob) of the duodenoscope 12. Here, the input of the switching instruction using the operation section 42 has been described, but this is merely an example. For example, the input may be an input via a foot switch (not illustrated) or a voice input via a microphone (not illustrated).

When the display control unit 82D receives the switching instruction via the external I/F 78, the display control unit 82D acquires another opening portion image 83 whose image size has been adjusted from the image adjustment unit 82C. The display control unit 82D updates the screen 36 to display the intestinal wall image 41 on which the other opening portion image 83 has been displayed, on the screen 36. In the example illustrated in FIG. 9, the opening portion pattern image 85A is switched to the opening portion pattern images 85B, 85C, and 85D in this order in response to the switching instruction. The physician 14 selects an appropriate opening portion image 83 (for example, an opening portion image 83 close to the opening portion expected in the preliminary consideration) by switching the opening portion image 83 while viewing the screen 36.

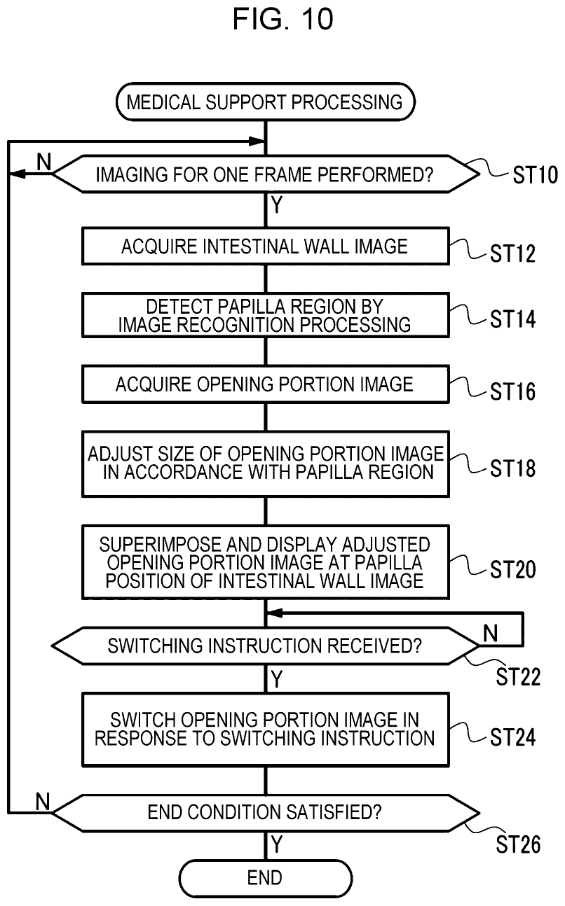

Next, an operation of a portion of the duodenoscope system 10 according to the technology of the present disclosure will be described with reference to FIG. 10.

FIG. 10 presents an example of the flow of medical support processing that is performed by the processor 82. The flow of the medical support processing presented in FIG. 10 is an example of a “medical support method” according to the technology of the present disclosure.

In the medical support processing presented in FIG. 10, first, in step ST10, the image acquisition unit 82A determines whether imaging for one frame has been performed by the camera 48 provided in the endoscopic scope 18. In step ST10, when the imaging for one frame has not been performed by the camera 48, the determination is denied, and the determination of step ST10 is performed again. In step ST10, when the imaging for one frame has been performed by the camera 48, the determination is allowed, and the medical support processing proceeds to step ST12.

In step ST12, the image acquisition unit 82A acquires an intestinal wall image 41 for one frame from the camera 48 provided in the endoscopic scope 18. After the processing of step ST12 is executed, the medical support processing proceeds to step ST14.

In step ST14, the image recognition unit 82B performs AI-based image recognition processing (that is, image recognition processing using the trained model 84B) on the intestinal wall image 41 acquired in step ST12, thereby detecting a papilla region N1. After the processing of step ST14 is executed, the medical support processing proceeds to step ST16.

In step ST16, the image adjustment unit 82C acquires an opening portion image 83 from the NVM 84. After the processing of step ST16 is executed, the medical support processing proceeds to step ST18.

In step ST18, the image adjustment unit 82C adjusts the size of the opening portion image 83 in accordance with the size of the papilla region N1. That is, the image adjustment unit 82C adjusts the size of the opening portion image 83 so that the opening portion indicated by the opening portion image 83 is displayed in the papilla region N1 in the intestinal wall image 41. After the processing of step ST18 is executed, the medical support processing proceeds to step ST20.

In step ST20, the display control unit 82D superimposes and displays the opening portion image 83 on the papilla region N1 in the intestinal wall image 41. After the processing of step ST20 is executed, the medical support processing proceeds to step ST22.

In step ST22, the display control unit 82D determines whether a switching instruction for switching the opening portion image 83 input by the physician 14 has been received. In step ST22, when the switching instruction is not received by the display control unit 82D, the determination is denied, and the processing of step ST22 is executed again. In step ST22, when the switching instruction has been received by the display control unit 82D, the determination is allowed, and the medical support processing proceeds to step ST24.

In step ST24, the display control unit 82D switches the opening portion image 83 in response to the switching instruction received in step ST22. After the processing of step ST24 is executed, the medical support processing proceeds to step ST26.

In step ST26, the display control unit 82D determines whether a condition for ending the medical support processing has been satisfied. An example of the condition for ending the medical support processing is a condition that an instruction for ending the medical support processing has been given to the duodenoscope system 10 (for example, a condition that the instruction for ending the medical support processing has been received by the reception device 62).

In step ST26, when the condition for ending the medical support processing has not been satisfied, the determination is denied, and the medical support processing proceeds to step ST10. In step ST26, when the condition for ending the medical support processing has been satisfied, the determination is allowed, and the medical support processing ends.

As described above, in the duodenoscope system 10 according to the first embodiment, the papilla region N1 is detected by the image recognition unit 82B executing the image recognition processing on the intestinal wall image 41 in the processor 82. Also, the intestinal wall image 41 is displayed on the screen 36 of the display device 13 by the display control unit 82D, and further, the opening portion image 83 simulating the opening portion existing in the papilla N is displayed in the papilla region N1 in the intestinal wall image 41. For example, in the ERCP examination using the duodenoscope 12, a procedure of inserting a cannula into the papilla N may be performed. In this case, the insertion position, the insertion angle, or the like of the cannula is adjusted in accordance with the position or the type of the opening portion in the papilla N. That is, the physician 14 inserts the cannula while checking the opening portion of the papilla N included in the intestinal wall image 41. In this configuration, the opening portion image 83 is displayed in the papilla region N1 of the intestinal wall image 41. Accordingly, the user such as the physician 14 can visually recognize the opening portion existing in the papilla N.

For example, in the ERCP examination, since the physician 14 concentrates on the operation of inserting the cannula, it is difficult for the physician 14 to memorize the type of the papilla N, the position of the opening portion in the intestinal wall image 41, or the like, or to refer to the information relating to the opening portion and displayed at a position other than the intestinal wall image 41. In this configuration, since the opening portion image 83 is displayed in the papilla region N1 of the intestinal wall image 41, the physician 14 can visually recognize the opening portion while performing the operation of inserting the cannula. As a result, the operation of inserting the cannula in the ERCP examination is facilitated.

Also, in the duodenoscope system 10, the opening portion image 83 includes the opening portion pattern image 85 selected in response to the switching instruction of the user from the plurality of opening portion pattern images 85 expressing different geometric features of the opening portion in the papilla N. In this configuration, the opening portion pattern image 85 designated as the result of the selection by the user among the plurality of opening portion pattern images 85 is displayed on the screen 36. Accordingly, the opening portion image 83 having a geometric feature close to the geometric feature intended by the user can be displayed on the screen. Also, for example, compared to a case where there is only one opening portion pattern image 85, it is possible to select the opening portion pattern image 85 having a geometric feature close to the geometric feature intended by the user.

Also, in the duodenoscope system 10, the plurality of opening portion pattern images 85 are displayed one by one on the screen 36, and the opening portion pattern image 85 displayed on the screen 36 is switched in response to the switching instruction by the user. Accordingly, the plurality of opening portion pattern images 85 can be displayed one by one at the timing intended by the user.

Also, in the duodenoscope system 10, the geometric feature of the opening portion is the position and/or the size of the opening portion in the papilla N. The position and/or the size of the opening portion varies depending on the type of the papilla N. In this configuration, the plurality of opening portion pattern images 85 having different positions and/or sizes of the opening portion in the papilla N are prepared. Accordingly, the opening portion image 83 having the position and/or the size of the opening portion close to the position and/or the size of the opening portion intended by the user can be displayed on the screen.

Also, in the duodenoscope system 10, the opening portion image 83 is a rendering image obtained by one or more modalities 11 and/or an image created based on finding information obtained from a finding input by the user. Accordingly, the opening portion image 83 close to the state of the actual opening portion can be displayed on the screen 36.

Also, in the duodenoscope system 10, the size of the opening portion image 83 changes in accordance with the size of the papilla region N1 in the screen 36. Accordingly, even when the size of the papilla region N1 changes, the size relationship between the papilla region N1 and the opening portion image 83 can be maintained.

Also, in the duodenoscope system 10, the opening portion consists of one or more openings. Accordingly, the user can visually recognize the opening portion existing in the papilla N regardless of whether the opening portion is one opening or a plurality of openings. First Modification

In the above-described first embodiment, the embodiment example in which the opening portion image 83 is the image indicating the opening portion in the papilla region N1 has been described, but the technology of the present disclosure is not limited thereto. In this first modification, an opening portion image 83 includes an existence probability map that is a map indicating the probability that the opening portion exists in the papilla N.

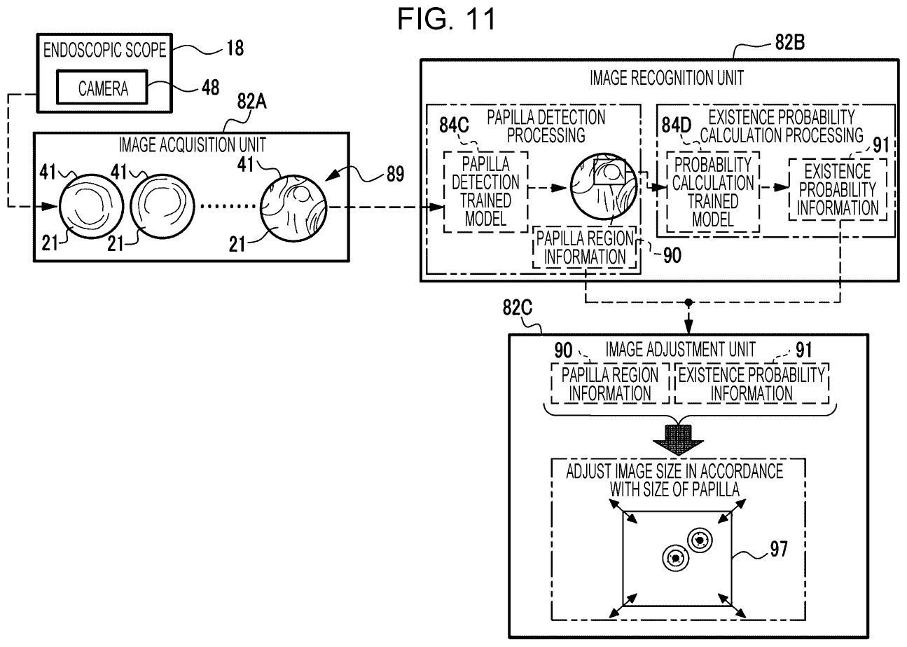

In one example, as illustrated in FIG. 11, the image acquisition unit 82A acquires an intestinal wall image 41 from the camera 48 provided in the endoscopic scope 18. The image acquisition unit 82A updates a time-series image group 89 by a FIFO method every time the image acquisition unit 82A acquires an intestinal wall image 41 from the camera 48.

The image recognition unit 82B performs papilla detection processing on the time-series image group 89 using a papilla detection trained model 84C. The image recognition unit 82B acquires the time-series image group 89 from the image acquisition unit 82A, and inputs the acquired time-series image group 89 to the papilla detection trained model 84C. Accordingly, the papilla detection trained model 84C outputs papilla region information 90 corresponding to the input time-series image group 89. The image recognition unit 82B acquires the papilla region information 90 output from the papilla detection trained model 84C.

The papilla detection trained model 84C is obtained by optimizing a neural network by performing machine learning on the neural network using training data. The training data is a plurality of data (that is, data of a plurality of frames) in which example data and correct answer data are associated with each other. The example data is, for example, an image (for example, an image corresponding to the intestinal wall image 41) obtained by imaging an area (for example, the inner wall of the duodenum) that can be a target of the ERCP examination. The correct answer data is an annotation corresponding to the example data. An example of the correct answer data is an annotation capable of specifying the papilla region N1.

The image recognition unit 82B performs existence probability calculation processing on the papilla region N1 indicated by the papilla region information 90. By performing the existence probability calculation processing, the existence probability of the opening portion in the papilla region N1 is calculated. In the present embodiment, the calculation of the existence probability of the opening portion represents processing of calculating a score indicating the probability that the opening portion exists for each pixel indicating the papilla region N1 and storing the score in the memory.

The image recognition unit 82B inputs an image indicating the papilla region N1 specified by the papilla detection processing to a probability calculation trained model 84D. Accordingly, the probability calculation trained model 84D outputs a score indicating the probability that the opening portion exists for each pixel in the input image indicating the papilla region N1. In other words, the probability calculation trained model 84D outputs existence probability information 91 that is information indicating the score for each pixel. The image recognition unit 82B acquires the existence probability information 91 output from the probability calculation trained model 84D.

The probability calculation trained model 84D is obtained by optimizing a neural network by performing machine learning on the neural network using training data. The training data is a plurality of data (that is, data of a plurality of frames) in which example data and correct answer data are associated with each other. The example data is, for example, an image (for example, an image corresponding to the intestinal wall image 41) obtained by imaging an area (for example, the inner wall of the duodenum) that can be a target of the ERCP examination. The correct answer data is an annotation corresponding to the example data. An example of the correct answer data is an annotation capable of specifying the opening portion.

Here, the embodiment example in which the papilla region N1 is detected using the papilla detection trained model 84C, and the existence probability of the opening portion in the papilla region N1 is calculated using the probability calculation trained model 84D has been described, but the technology of the present disclosure is not limited thereto. For example, one trained model that detects the papilla region N1 and calculates the existence probability of the opening portion may be used for the intestinal wall image 41. Also, a trained model that calculates the existence probability of the opening portion for the entirety of the intestinal wall image 41 may be used.

The image adjustment unit 82C generates an existence probability map 97 based on the existence probability information 91. The existence probability map 97 is an example of a “map” according to the technology of the present disclosure. The existence probability map 97 is an image having a score indicating the existence probability of the opening portion as a pixel value. For example, the existence probability map 97 is an image in which RGB values (that is, red (R), green (G), and blue (B)) of each pixel are changed in accordance with the score that is the pixel value. Also, the image adjustment unit 82C adjusts the size of the existence probability map 97 in accordance with the size of the papilla N indicated by the papilla region information 90.

Here, the example in which the RGB value of each pixel is changed has been described as the existence probability map 97, but this is merely an example. For example, as the existence probability map 97, the transparency may be changed in accordance with the score. Alternatively, as the existence probability map 97, a region whose score is a predetermined value or more may be displayed in a manner in which the region can be distinguished from other regions (for example, a manner in which a color is changed or blinking is provided, or the like).

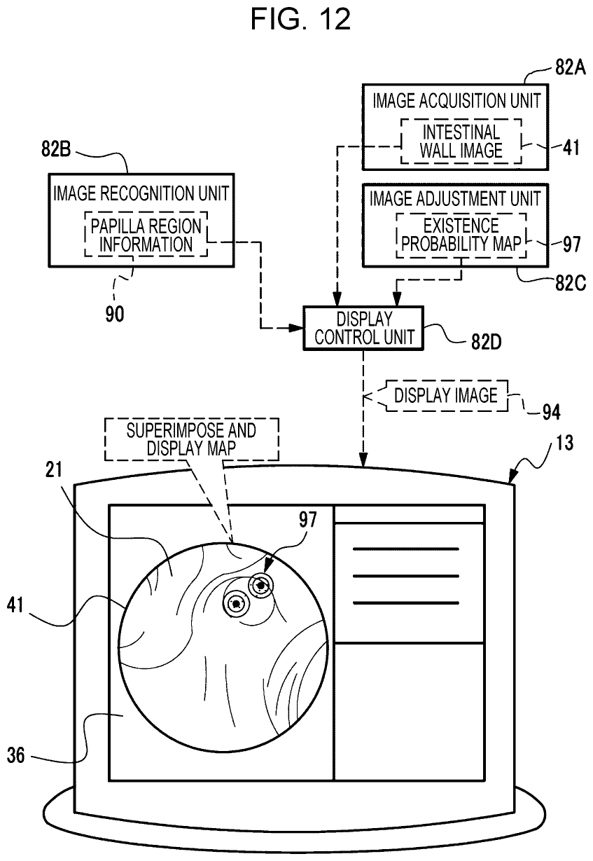

In one example, as illustrated in FIG. 12, the display control unit 82D acquires an intestinal wall image 41 from the image acquisition unit 82A. Also, the display control unit 82D acquires papilla region information 90 from the image recognition unit 82B. Further, the display control unit 82D acquires an existence probability map 97 from the image adjustment unit 82C. Here, the image size of the existence probability map 97 is adjusted by the image adjustment unit 82C in accordance with the size of the papilla region N1.

The display control unit 82D superimposes and displays the existence probability map 97 in the papilla region N1 in the intestinal wall image 41. To be specific, the display control unit 82D displays the existence probability map 97 whose image size has been adjusted at the position of the papilla region N1 indicated by the papilla region information 90 in the intestinal wall image 41. Accordingly, the existence probability of the opening portion indicated by the existence probability map 97 is displayed in the papilla region N1 in the intestinal wall image 41. Further, the display control unit 82D performs GUI control for displaying a display image 94 including the intestinal wall image 41, thereby causing the display device 13 to display the screen 36. For example, the physician 14 visually recognizes the existence probability map 97 displayed on the screen 36 and uses the existence probability map 97 as a guide for inserting a cannula into the papilla N.

As described above, in the duodenoscope system 10 according to this first modification, the existence probability map 97 is displayed as the opening portion image 83 in the intestinal wall image 41. The existence probability map 97 is an image indicating the distribution of the probability that the opening portion exists in the papilla region N1 in the intestinal wall image 41. Accordingly, the user can accurately grasp a region having a high probability that the opening portion exists in the papilla region N1 in the intestinal wall image 41.

Also, in the duodenoscope system 10, the AI-based image recognition processing is performed on the intestinal wall image 41, and the distribution of the probability that the opening portion exists is obtained by the image recognition processing being executed. Accordingly, it is possible to easily obtain the distribution of the probability that the opening portion exists in the papilla region N1 in the intestinal wall image 41.

### Second Embodiment

In the above-described first embodiment, the embodiment example in which the opening portion image 83 is superimposed and displayed on the intestinal wall image 41 has been described, but the technology of the present disclosure is not limited thereto. In this second embodiment, a duct path image 95 is superimposed and displayed on the intestinal wall image 41. The duct path image 95 is an image indicating the paths of the bile duct and the pancreatic duct. The duct path image 95 is an example of a “duct path image” according to the technology of the present disclosure.

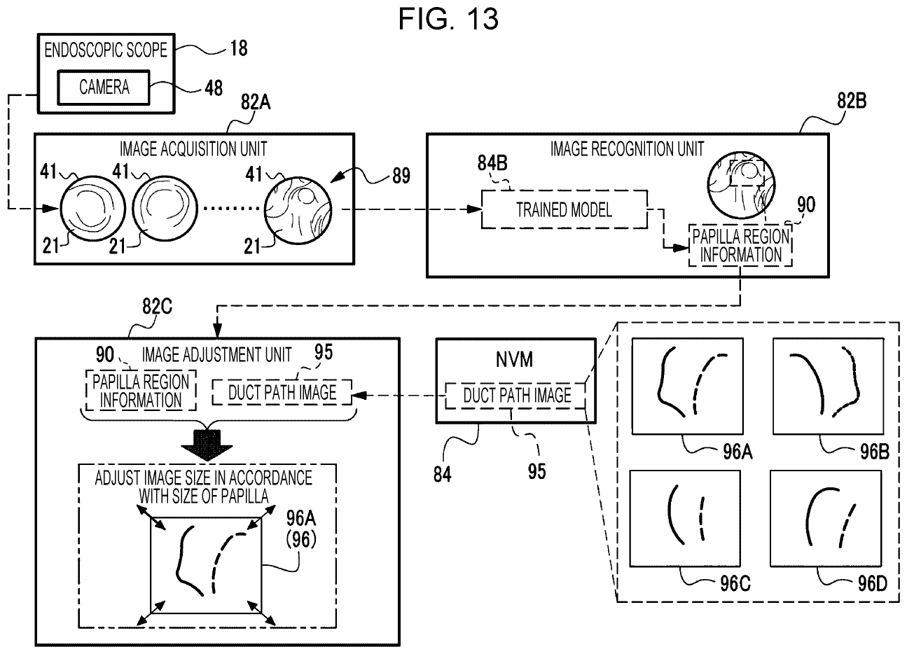

In one example, as illustrated in FIG. 13, the image acquisition unit 82A acquires an intestinal wall image 41 from the camera 48 provided in the endoscopic scope 18. The image acquisition unit 82A updates a time-series image group 89 by a FIFO method every time the image acquisition unit 82A acquires an intestinal wall image 41 from the camera 48.

The image recognition unit 82B performs image recognition processing on the time-series image group 89 using the trained model 84B. The image recognition unit 82B acquires the time-series image group 89 from the image acquisition unit 82A, and inputs the acquired time-series image group 89 to the trained model 84B. Accordingly, the trained model 84B outputs papilla region information 90 corresponding to the input time-series image group 89. The image recognition unit 82B acquires the papilla region information 90 output from the trained model 84B.

The image adjustment unit 82C acquires the papilla region information 90 from the image recognition unit 82B. Also, the image adjustment unit 82C acquires a duct path image 95 from the NVM 84. The duct path image 95 includes a plurality of path pattern images 96A to 96D. In the following description, when the plurality of path pattern images 96A to 96D are not distinguished from each other, they are simply referred to as “path pattern images 96”. The plurality of path pattern images 96 are images expressing geometric features of the pancreatic duct and the bile duct in the intestinal wall. Here, the geometric features of the bile duct and the pancreatic duct represent the positions and/or the sizes of the paths of the bile duct and the pancreatic duct in the intestinal wall. That is, the plurality of path pattern images 96 are different from each other in the positions and/or the sizes of the bile duct and the pancreatic duct. Here, the example in which the four path pattern images 96A to 96D are included in the duct path image 95 has been described, but this is merely an example, and the number of images included in the duct path image 95 may be two or three, or may be five or more. The path pattern image 96 is an example of a “second pattern image” according to the technology of the present disclosure.

Also, here, the embodiment example in which both the path of the bile duct and the path of the pancreatic duct are indicated as the duct path image 95 has been described, but the technology of the present disclosure is not limited thereto. The duct path image 95 may be an image indicating only the path of the bile duct, or may be an image indicating only the path of the pancreatic duct.

The image adjustment unit 82C adjusts the size of the duct path image 95 in accordance with the size of the papilla region N1 indicated by the papilla region information 90. The image adjustment unit 82C adjusts the size of the duct path image 95 using, for example, an adjustment table (not illustrated). The adjustment table is a table in which the size of the papilla region N1 is set as an input value and the size of the duct path image 95 is set as an output value. By enlarging or reducing the duct path image 95, the size of the duct path image 95 is adjusted.

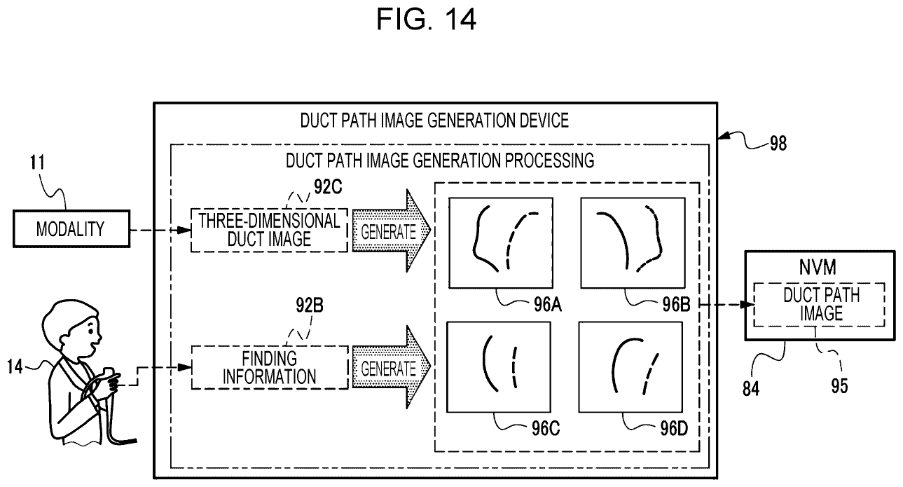

In one example, as illustrated in FIG. 14, the duct path image 95 is generated by a duct path image generation device 98. The duct path image generation device 98 is an external device connectable to the image processing device 25. The hardware configuration (for example, a processor, a NVM, a RAM, and the like) of the duct path image generation device 98 is basically the same as the hardware configuration of the control device 22 illustrated in FIG. 3, and hence the description relating to the hardware configuration of the duct path image generation device 98 will be omitted here.

In the duct path image generation device 98, duct path image generation processing is executed. In the duct path image generation processing, a three-dimensional duct image 92C is generated based on volume data obtained by a modality 11 (for example, a CT apparatus or a MRI apparatus). The three-dimensional duct image 92C is an example of a “second reference image” according to the technology of the present disclosure. Further, rendering by viewing the three-dimensional duct image 92C from a predetermined viewpoint (for example, a viewpoint directly facing the papilla) is performed, thereby generating a duct path image 95.

Also, in the duct path image generation processing, a duct path image 95 is generated based on finding information 92B input by the physician 14 via the reception device 62. The finding information 92B is an example of “second information” according to the technology of the present disclosure. Here, the finding information 92B is information indicating the position, the shape, and/or the size of the duct path designated by the user. To be specific, for example, the physician 14 inputs the finding information 92B by designating the positions, the shapes, and the sizes of the bile duct and the pancreatic duct using a keyboard as the reception device 62. In another example, finding information 92B is generated based on a statistical value (for example, the mode) of position coordinates of a region diagnosed as the paths of the bile duct and the pancreatic duct in a past examination. The duct path image generation device 98 outputs a plurality of path pattern images 96 generated in the duct path image generation processing to the NVM 84 of the image processing device 25 as the duct path image 95.

Here, the embodiment example in which the duct path image 95 is generated in the duct path image generation device 98 has been described, but the technology of the present disclosure is not limited thereto. For example, an aspect in which the image processing device 25 has a function equivalent to that of the duct path image generation device 98, and the duct path image 95 is generated in the image processing device 25 may be employed.

Also, here, the embodiment example in which the duct path image 95 is generated from the three-dimensional duct image 92C and the finding information 92B has been described, but the technology of the present disclosure is not limited thereto. For example, the duct path image 95 may be generated from any one of the three-dimensional duct image 92C and the finding information 92B.

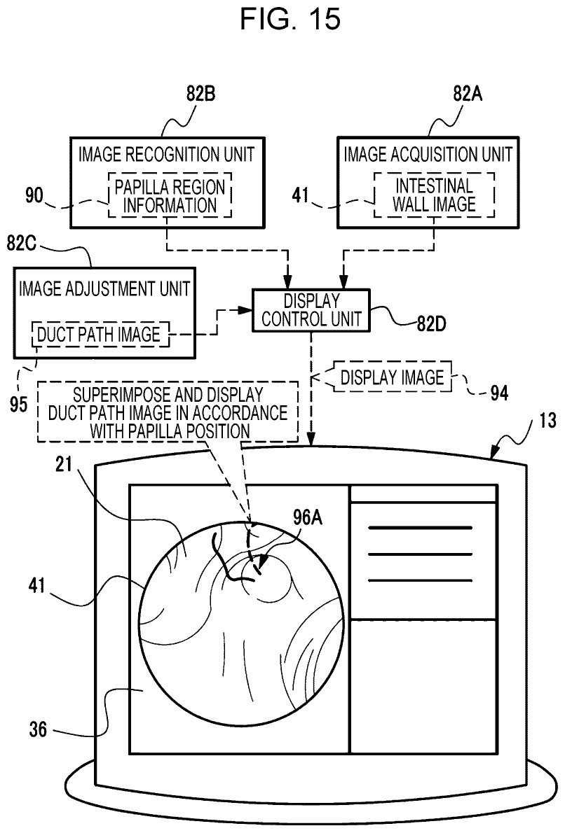

In one example, as illustrated in FIG. 15, the display control unit 82D acquires an intestinal wall image 41 from the image acquisition unit 82A. Also, the display control unit 82D acquires papilla region information 90 from the image recognition unit 82B. Further, the display control unit 82D acquires a duct path image 95 from the image adjustment unit 82C. Here, the image size of the duct path image 95 has been adjusted by the image adjustment unit 82C in accordance with the size of the papilla region N1.

The display control unit 82D superimposes and displays the duct path image 95 in accordance with the papilla region N1 in the intestinal wall image 41. To be specific, the display control unit 82D displays the duct path image 95 whose image size has been adjusted so that end portions of the bile duct and the pancreatic duct indicated by the duct path image 95 are positioned in the papilla region N1 indicated by the papilla region information 90 in the intestinal wall image 41. Accordingly, the paths of the bile duct and the pancreatic duct indicated by the duct path image 95 are displayed in the intestinal wall image 41. Further, the display control unit 82D generates a display image 94 including the intestinal wall image 41 on which the duct path image 95 has been superimposed and outputs the display image 94 to the display device 13. In the example illustrated in FIG. 15, the path pattern image 96A is superimposed and displayed on the intestinal wall image 41. For example, the physician 14 visually recognizes the path pattern image 96A displayed on the screen 36 and uses the path pattern image 96A as a guide for intubating a cannula into the bile duct or the pancreatic duct. Note that the path pattern image 96 to be displayed first may be determined in advance, or may be designated by the user.

Also, when the intestinal wall image 41 is displayed in an enlarged or reduced manner by the user's operation, the duct path image 95 is also enlarged or reduced in accordance with the enlargement or the reduction of the intestinal wall image 41. In this case, the image adjustment unit 82C adjusts the size of the duct path image 95 in accordance with the size of the intestinal wall image 41. Then, the display control unit 82D superimposes and displays the duct path image 95 whose size has been adjusted on the intestinal wall image 41.

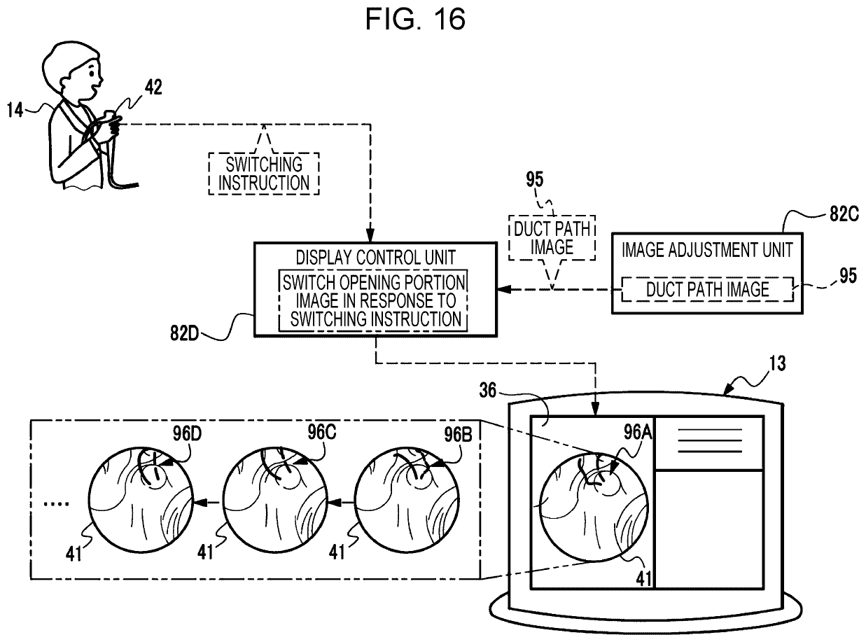

In one example, as illustrated in FIG. 16, the display control unit 82D performs processing of switching in response to a switching instruction from the physician 14. The physician 14 inputs the switching instruction of the duct path image 95, for example, via the operation section 42 (for example, an operation knob) of the duodenoscope 12. When the display control unit 82D receives the switching instruction via the external I/F 78, the display control unit 82D acquires another duct path image 95 whose image size has been adjusted from the image adjustment unit 82C. The display control unit 82D updates the screen 36 to display the intestinal wall image 41 on which the other duct path image 95 is displayed, on the screen 36. In the example illustrated in FIG. 16, the duct path image 95 is switched to the path pattern images 96B, 96C, and 96D in this order in response to the switching instruction. The physician 14 selects an appropriate duct path image 95 (for example, a duct path image 95 close to the opening portion expected in the preliminary consideration) by switching the duct path image 95 while viewing the screen 36.

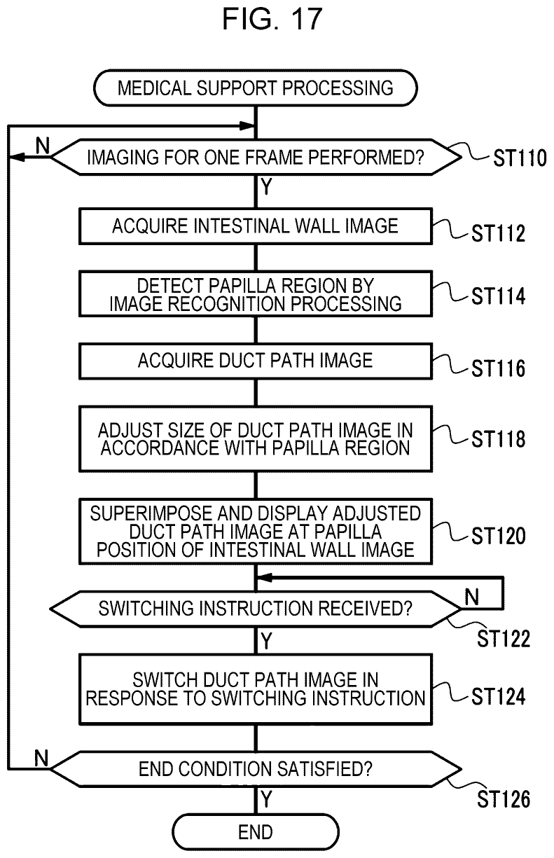

Next, an operation of a portion of the duodenoscope system 10 according to the technology of the present disclosure will be described with reference to FIG. 17.

FIG. 17 presents an example of the flow of medical support processing that is performed by the processor 82. The flow of the medical support processing presented in FIG. 17 is an example of a “medical support method” according to the technology of the present disclosure.

In the medical support processing presented in FIG. 17, first, in step ST110, the image acquisition unit 82A determines whether imaging for one frame has been performed by the camera 48 provided in the endoscopic scope 18. In step ST110, when the imaging for one frame has not been performed by the camera 48, the determination is denied, and the determination of step ST110 is performed again. In step ST110, when the imaging for one frame has been performed by the camera 48, the determination is allowed, and the medical support processing proceeds to step ST112.

In step ST112, the image acquisition unit 82A acquires an intestinal wall image 41 for one frame from the camera 48 provided in the endoscopic scope 18. After the processing of step ST112 is executed, the medical support processing proceeds to step ST114.

In step ST114, the image recognition unit 82B performs AI-based image recognition processing (that is, image recognition processing using the trained model 84B) on the intestinal wall image 41 acquired in step ST112, thereby detecting a papilla region N1. After the processing of step ST114 is executed, the medical support processing proceeds to step ST116.

In step ST116, the image adjustment unit 82C acquires a duct path image 95 from the NVM 84. After the processing of step ST116 is executed, the medical support processing proceeds to step ST118.

In step ST118, the image adjustment unit 82C adjusts the size of the duct path image 95 in accordance with the size of the papilla region N1. That is, the image adjustment unit 82C adjusts the size of the duct path image 95 so that the paths of the bile duct and the pancreatic duct are displayed in the intestinal wall image 41. After the processing of step ST118 is executed, the medical support processing proceeds to step ST120.

In step ST120, the display control unit 82D superimposes and displays the duct path image 95 on the papilla region N1 in the intestinal wall image 41. After the processing of step ST120 is executed, the medical support processing proceeds to step ST122.

In step ST122, the display control unit 82D determines whether a switching instruction for switching the duct path image 95 input by the physician 14 has been received. In step ST122, when the switching instruction is not received by the display control unit 82D, the determination is denied, and the processing of step ST122 is executed again. In step ST122, when the switching instruction has been received by the display control unit 82D, the determination is allowed, and the medical support processing proceeds to step ST124.

In step ST124, the display control unit 82D switches the duct path image 95 in response to the switching instruction received in step ST122. After the processing of step ST124 is executed, the medical support processing proceeds to step ST126.

In step ST126, the display control unit 82D determines whether a condition for ending the medical support processing has been satisfied. An example of the condition for ending the medical support processing is a condition that an instruction for ending the medical support processing has been given to the duodenoscope system 10 (for example, a condition that the instruction for ending the medical support processing has been received by the reception device 62).

In step ST126, when the condition for ending the medical support processing has not been satisfied, the determination is denied, and the medical support processing proceeds to step ST110. In step ST126, when the condition for ending the medical support processing has been satisfied, the determination is allowed, and the medical support processing ends.

As described above, in the duodenoscope system 10 according to the second embodiment, the papilla region N1 is detected by the image recognition unit 82B executing the image recognition processing on the intestinal wall image 41 in the processor 82. Also, the intestinal wall image 41 is displayed on the screen 36 of the display device 13 by the display control unit 82D, and further, the duct path image 95 indicating the duct paths of the bile duct and the pancreatic duct is displayed in the intestinal wall image 41. For example, in the ERCP examination using the duodenoscope 12, a procedure of intubating a cannula into the bile duct or the pancreatic duct may be performed. In this case, the direction in which the cannula is inserted, the length of the cannula to be inserted, or the like is adjusted in accordance with the path of the bile duct or the pancreatic duct. That is, the physician 14 inserts the cannula while predicting the path of the bile duct or the pancreatic duct. In this configuration, the duct path image 95 is displayed in the intestinal wall image 41. Accordingly, the user such as the physician 14 can visually recognize the path of the pancreatic duct or the bile duct.

For example, in the ERCP examination, since the physician 14 concentrates on the operation of intubating the cannula, it is difficult for the physician 14 to memorize the paths of the bile duct and the pancreatic duct, or to refer to the information relating to the bile duct and the pancreatic duct and displayed at a position other than the intestinal wall image 41. In this configuration, since the duct path image 95 is displayed in the intestinal wall image 41, the physician 14 can visually recognize the paths of the bile duct and the pancreatic duct while performing the operation of inserting the cannula. As a result, the operation of intubating the cannula in the ERCP examination is facilitated.

Also, in the duodenoscope system 10, the duct path image 95 includes the path pattern image 96 selected in response to the switching instruction of the user from the plurality of path pattern images 96 expressing different geometric features of the bile duct and the pancreatic duct. In this configuration, the path pattern image 96 designated as the result of the selection by the user among the plurality of path pattern images 96 is displayed on the screen 36. Accordingly, the duct path image 95 having a geometric feature close to the geometric feature intended by the user can be displayed on the screen. Also, for example, compared to a case where there is only one path pattern image 96, it is possible to select the path pattern image 96 having a geometric feature close to the geometric feature intended by the user.

Also, in the duodenoscope system 10, the plurality of path pattern images 96 are displayed one by one on the screen 36, and the path pattern image 96 displayed on the screen 36 is switched in response to the switching instruction by the user. Accordingly, the plurality of path pattern images 96 can be displayed one by one at the timing intended by the user.

Also, in the duodenoscope system 10, the geometric features of the bile duct and the pancreatic duct are the positions and/or the sizes of the bile duct and the pancreatic duct in the intestinal wall. In this configuration, the plurality of path pattern images 96 having different positions and/or sizes of the bile duct and the pancreatic duct in the intestinal wall are prepared. Accordingly, the duct path image 95 having the positions and/or the sizes of the bile duct and the pancreatic duct close to the positions and/or the sizes of the bile duct and the pancreatic duct intended by the user can be displayed on the screen.

Also, in the duodenoscope system 10, the duct path image 95 is a rendering image obtained by one or more modalities 11 and/or an image created based on finding information obtained from a finding input by the user. Accordingly, the duct path image 95 close to the state of the actual bile duct and pancreatic duct can be displayed on the screen 36.

### Second Modification

In the above-described second embodiment, the embodiment example in which the duct path image 95 is displayed in accordance with the detection result of the papilla N has been described, but the technology of the present disclosure is not limited thereto. In this second modification, the duct path image 95 is displayed in the intestinal wall image 41 in accordance with the existence probability of the opening portion in the papilla region N1.

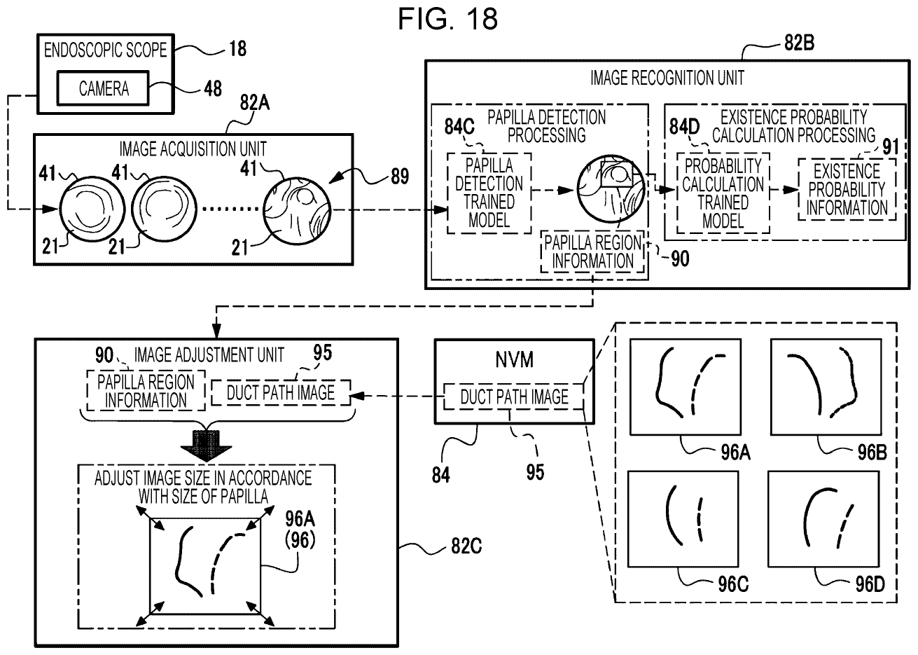

In one example, as illustrated in FIG. 18, the image acquisition unit 82A acquires an intestinal wall image 41 from the camera 48 provided in the endoscopic scope 18. The image acquisition unit 82A updates a time-series image group 89 by a FIFO method every time the image acquisition unit 82A acquires an intestinal wall image 41 from the camera 48.

The image recognition unit 82B performs papilla detection processing on the time-series image group 89 using a papilla detection trained model 84C. The image recognition unit 82B acquires the time-series image group 89 from the image acquisition unit 82A, and inputs the acquired time-series image group 89 to the papilla detection trained model 84C. Accordingly, the papilla detection trained model 84C outputs papilla region information 90 corresponding to the input time-series image group 89. The image recognition unit 82B acquires the papilla region information 90 output from the papilla detection trained model 84C.

The image recognition unit 82B performs existence probability calculation processing on the papilla region N1 indicated by the papilla region information 90. By performing the existence probability calculation processing, the existence probability of the opening portion in the papilla region N1 is calculated.

The image recognition unit 82B inputs an image indicating the papilla region N1 specified by the papilla detection processing to a probability calculation trained model 84D. Accordingly, the probability calculation trained model 84D outputs a score indicating the probability that the opening portion exists for each pixel in the input image indicating the papilla region N1. In other words, the probability calculation trained model 84D outputs existence probability information 91 that is information indicating the score for each pixel. The image recognition unit 82B acquires the existence probability information 91 output from the probability calculation trained model 84D.

The image adjustment unit 82C acquires the papilla region information 90 from the image recognition unit 82B. Also, the image adjustment unit 82C acquires a duct path image 95 from the NVM 84. The image adjustment unit 82C adjusts the size of the duct path image 95 in accordance with the size of the papilla region N1 indicated by the papilla region information 90. Accordingly, by enlarging or reducing the duct path image 95, the size of the duct path image 95 is adjusted.

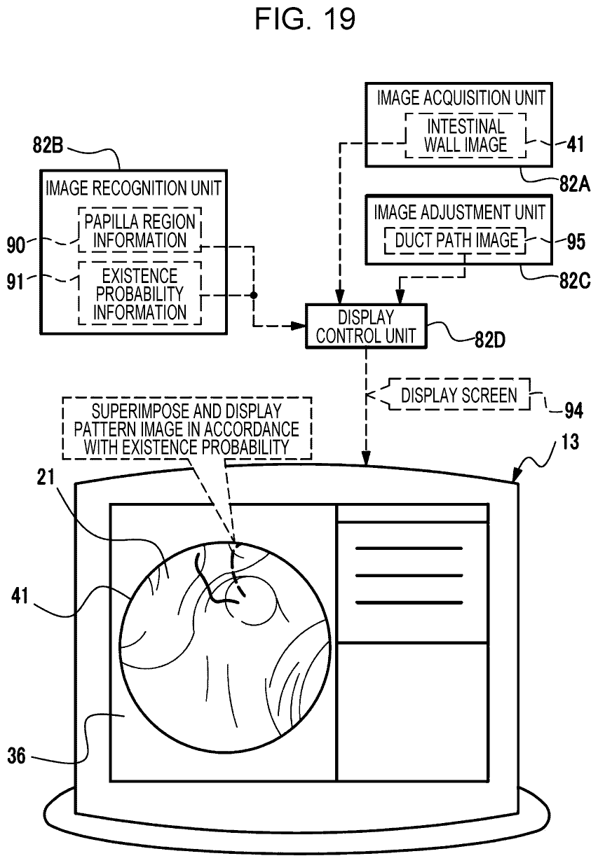

In one example, as illustrated in FIG. 19, the display control unit 82D acquires an intestinal wall image 41 from the image acquisition unit 82A. Also, the display control unit 82D acquires papilla region information 90 and existence probability information 91 from the image recognition unit 82B. Further, the display control unit 82D acquires a duct path image 95 from the image adjustment unit 82C.

The display control unit 82D superimposes and displays the duct path image 95 based on the existence probability information 91 in the intestinal wall image 41. To be specific, the display control unit 82D displays the duct path image 95 so that end portions of the bile duct and the pancreatic duct indicated by the duct path image 95 are positioned in a region where the existence probability of the opening portion indicated by the existence probability information 91 exceeds a predetermined value in the intestinal wall image 41. Further, the display control unit 82D performs GUI control for displaying a display image 94 including the intestinal wall image 41, thereby causing the display device 13 to display the screen 36.

As described above, in the duodenoscope system 10 according to this second modification, the duct path image 95 indicating the duct paths of the bile duct and the pancreatic duct is displayed in the intestinal wall image 41 based on the existence probability information 91 obtained by the image recognition processing on the intestinal wall image 41. Accordingly, it is possible to display the duct path image 95 at a more accurate position.

### Third Embodiment

In the above-described first embodiment and the above-described second embodiment, the embodiment example in which the opening portion image 83 or the duct path image 95 is superimposed and displayed on the intestinal wall image 41 has been described, but the technology of the present disclosure is not limited thereto. In this third embodiment, the opening portion image 83 and the duct path image 95 are superimposed and displayed on the intestinal wall image 41.

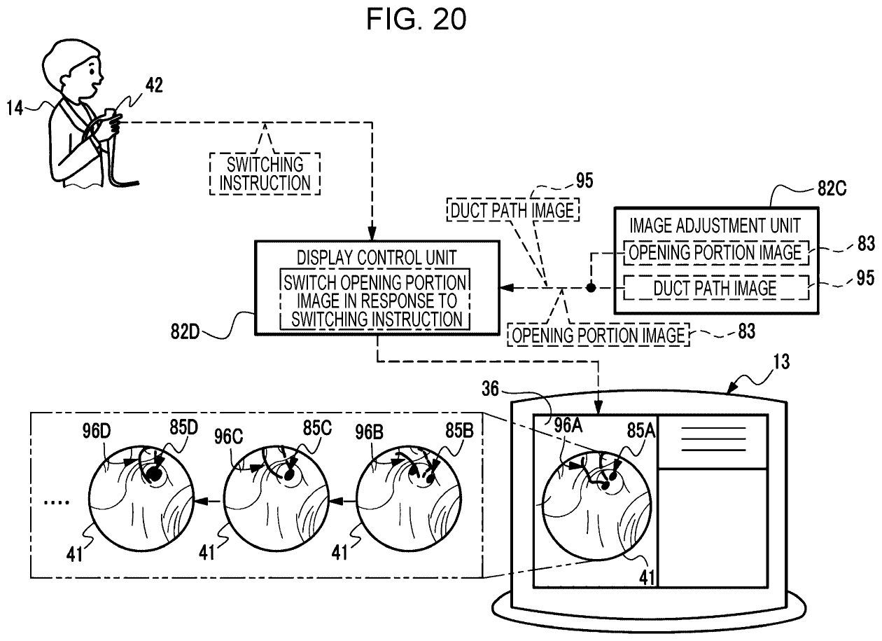

In one example, as illustrated in FIG. 20, the display control unit 82D superimposes and displays an opening portion image 83 and a duct path image 95 in a papilla region N1 in an intestinal wall image 41. Accordingly, the opening portion indicated by the opening portion image 83 and the paths of the bile duct and the pancreatic duct indicated by the duct path image 95 are displayed in the intestinal wall image 41.

The display control unit 82D performs processing of switching the opening portion image 83 and the duct path image 95 in response to a switching instruction from the physician 14. When the display control unit 82D receives the switching instruction via the external I/F 78, the image adjustment unit 82C acquires, from the NVM 84, an opening portion image 83 and a duct path image 95 different from the opening portion image 83 and the duct path image 95 currently displayed. Then, the image adjustment unit 82C adjusts the image sizes of the opening portion image 83 and the duct path image 95.

The display control unit 82D acquires the opening portion image 83 and the duct path image 95 whose image sizes have been adjusted from the image adjustment unit 82C. The display control unit 82D superimposes and displays the opening portion image 83 and the duct path image 95 in the intestinal wall image 41, and further updates the screen 36. In the example illustrated in FIG. 20, there is provided an example in which the opening portion image 83 is switched to opening portion pattern images 85B, 85C, and 85D in this order in response to the switching instruction. There is provided an example in which the duct path image 95 is switched to path pattern images 96B, 96C, and 96D in this order in response to the switching instruction. The physician 14 selects appropriate opening portion pattern image 85 and path pattern image 96 by switching the images while viewing the screen 36.

Here, the embodiment example in which the opening portion image 83 and the duct path image 95 are simultaneously switched has been described, but the technology of the present disclosure is not limited thereto. The opening portion image 83 and the duct path image 95 may be independently switched.

As described above, in the duodenoscope system 10 according to this third embodiment, the opening portion image 83 and the duct path image 95 are displayed in the intestinal wall image 41. Accordingly, the user such as the physician 14 can visually recognize the position of the opening portion, and the path of the pancreatic duct or the bile duct.

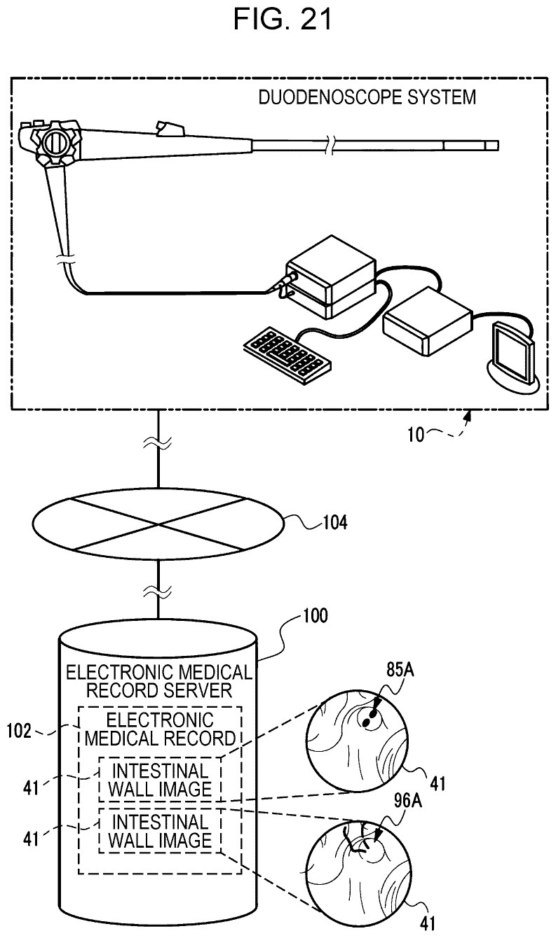

In each of the above-described embodiments, the embodiment example in which the intestinal wall image 41 on which the opening portion image 83 and/or the duct path image 95 is superimposed and displayed is output to the display device 13 and the intestinal wall image 41 is displayed on the screen 36 of the display device 13 has been described, but the technology of the present disclosure is not limited thereto. In one example, as illustrated in FIG. 21, an aspect in which the intestinal wall image 41 on which the opening portion image 83 and/or the duct path image 95 is superimposed and displayed is output to an electronic medical record server 100 may be employed. The electronic medical record server 100 is a server for storing electronic medical record information 102 indicating a result of medical diagnosis and treatment for a patient. The electronic medical record information 102 includes the intestinal wall image 41.

The electronic medical record server 100 is connected to the duodenoscope system 10 via a network 104. The electronic medical record server 100 acquires the intestinal wall image 41 from the duodenoscope system 10. The electronic medical record server 100 stores the intestinal wall image 41 as a portion of the result of medical diagnosis and treatment indicated by the electronic medical record information 102. In the example illustrated in FIG. 21, as the intestinal wall image 41, an intestinal wall image 41 on which an opening portion image 83 is superimposed and displayed and an intestinal wall image 41 on which a duct path image 95 is superimposed are illustrated. The electronic medical record server 100 is an example of an “external device” according to the technology of the present disclosure, and the electronic medical record information 102 is an example of a “medical record” according to the technology of the present disclosure.

The electronic medical record server 100 is also connected to a terminal other than the duodenoscope system 10 (for example, a personal computer installed in a medical facility) via the network 104. The user such as the physician 14 can obtain the intestinal wall image 41 stored in the electronic medical record server 100 via the terminal. As described above, since the intestinal wall image 41 including the opening portion image 83 and/or the duct path image 95 is stored in the electronic medical record server 100, the user can obtain the intestinal wall image 41 including the opening portion image 83 and/or the duct path image 95.

Also, in each of the above-described embodiments, the embodiment example in which the opening portion image 83 and/or the duct path image 95 is superimposed and displayed in the intestinal wall image 41 has been described, but the technology of the present disclosure is not limited thereto. The opening portion image 83 and/or the duct path image 95 may be embedded and displayed in the intestinal wall image 41.

Also, in each of the above-described embodiments, the embodiment example in which the papilla region N1 is detected by the AI-based image recognition processing in the intestinal wall image 41 has been described, but the technology of the present disclosure is not limited thereto. For example, the papilla region N1 may be detected by pattern-matching-based image recognition processing.

Also, in each of the above-described embodiments, the embodiment example in which the opening portion image 83 and the duct path image 95 are template images created in advance has been described, but the technology of the present disclosure is not limited thereto. The opening portion image 83 and the duct path image 95 may be changed or added in accordance with, for example, an input of the user.

Also, in each of the above-described embodiments, the embodiment example in which the opening portion image 83 and the duct path image 95 are displayed by the display control unit 82D in accordance with the position of the papilla region N1 detected by the image recognition processing has been described, but the technology of the present disclosure is not limited thereto. For example, the positions of the opening portion image 83 and the duct path image 95 with respect to the display result by the display control unit 82D may be adjusted in accordance with an input by the user.

Also, in each of the above-described embodiments, the embodiment example in which the moving image constituted by including the plurality of frames of the intestinal wall images 41 is displayed on the screen 36, and the opening portion image 83 and/or the duct path image 95 is superimposed and displayed on the intestinal wall image 41 has been described, but the technology of the present disclosure is not limited thereto. For example, an aspect in which an intestinal wall image 41 that is a still image of a designated frame (for example, a frame when an imaging instruction is input by the user) is displayed on a screen different from the screen 36, and the opening portion image 83 and/or the duct path image 95 is superimposed and displayed on the intestinal wall image 41 displayed on the different screen may be employed.

In the above-described embodiments, the embodiment example in which the medical support processing is performed by the processor 82 of the computer 76 included in the image processing device 25 has been described, but the technology of the present disclosure is not limited thereto. For example, the medical support processing may be performed by the processor 70 of the computer 64 included in the control device 22. Alternatively, the device that performs the medical support processing may be provided outside the duodenoscope 12. Examples of the device provided outside the duodenoscope 12 include at least one server and/or at least one personal computer or the like that is communicably connected to the duodenoscope 12. Alternatively, the medical support processing may be performed by a plurality of devices in a distributed manner.

In the above-described embodiments, the embodiment example in which the medical support processing program 84A is stored in the NVM 84 has been described, but the technology of the present disclosure is not limited thereto. For example, the medical support processing program 84A may be stored in a portable non-transitory storage medium such as a SSD or a USB memory. The medical support processing program 84A stored in the non-transitory storage medium is installed in the computer 76 of the duodenoscope 12. The processor 82 executes the medical support processing in accordance with the medical support processing program 84A.

Alternatively, the medical support processing program 84A may be stored in a storage device such as another computer or a server connected to the duodenoscope 12 via a network, and the medical support processing program 84A may be downloaded in response to a request from the duodenoscope 12 and installed in the computer 76.

Note that it is not necessary to store the entirety of the medical support processing program 84A in a storage device such as the other computer or the server device connected to the duodenoscope 12, or in the NVM 84, and a portion of the medical support processing program 84A may be stored.

As hardware resources for executing the medical support processing, the following various processors can be used. The processor may be, for example, a CPU that is a general-purpose processor that functions as a hardware resource for executing the medical support processing by executing software, that is, a program. Alternatively, the processor may be, for example, a dedicated electric circuit that is a processor, such as a FPGA, a PLD, or an ASIC, having a circuit configuration designed exclusively for executing specific processing. A memory is built in or connected to any one of the processors, and any one of the processors executes medical support processing using the memory.

The hardware resource that executes the medical support processing may be constituted of one of these various processors, or may be constituted of a combination of two or more processors of the same type or different types (for example, a combination of a plurality of FPGAs or a combination of a CPU and a FPGA). Alternatively, the hardware resource for executing the medical support processing may be one processor.

As an example of being constituted of one processor, first, there is an embodiment in which one processor is constituted of a combination of one or more CPUs and software, and this processor functions as the hardware resource for executing the medical support processing. Second, there is an embodiment of using a processor that implements the functions of the entire system including a plurality of hardware resources for executing the medical support processing by one IC chip, as typified by a SoC or the like. As described above, the medical support processing is implemented using one or more of the above-described various processors as the hardware resource.

Further, as a hardware structure of these various processors, more specifically, an electric circuit obtained by combining circuit elements such as semiconductor elements can be used. Also, the above-described medical support processing is merely an example. Thus, it is clear that unnecessary steps may be deleted, new steps may be added, or the processing order may be changed without departing from the scope.

The written contents and the illustrated contents given above are detailed description of portions according to the technology of the present disclosure, and are merely examples of the technology of the present disclosure. For example, the description relating to the above-described configurations, functions, operations, and effects is description relating to examples of the configurations, functions, operations, and effects of the portions according to the technology of the present disclosure. Hence, it is clear that unnecessary portions may be deleted, new elements may be added, or replacement may be performed on the written contents and the illustrated contents given above without departing from the scope of the technology of the present disclosure. Also, in order to avoid complexity and to facilitate understanding of the portions according to the technology of the present disclosure, description relating to common general technical knowledge and the like that do not particularly require description for enabling the technology of the present disclosure to be implemented in the written contents and the illustrated contents given above is omitted.

In this specification, “A and/or B” is synonymous with “at least one of A or B”. That is, “A and/or B” means that A alone may be present, B alone may be present, or a combination of A and B may be present. Also, in this specification, when three or more matters are combined and expressed by “and/or”, the same idea as “A and/or B” is applied.

All documents, patent applications, and technical standards mentioned in this specification are incorporated herein by reference to the same extent as if each individual document, patent application, or technical standard was specifically and individually indicated to be incorporated by reference.

JP2022-177611 filed on Nov. 4, 2022 is incorporated in the present specification by reference in its entirety.

What is claimed is:

1. A medical support device comprising:
a processor,
wherein the processor is configured to:
detect a duodenal papilla region by executing image recognition processing on an intestinal wall image obtained by a camera provided in an endoscopic scope imaging an intestinal wall of a duodenum;
display the intestinal wall image on a screen;
display an opening portion image simulating an opening portion existing in a duodenal papilla, in the duodenal papilla region in the intestinal wall image displayed on the screen; and
wherein the opening portion image is at least one of a plurality of first pattern images expressing different first geometric features of the opening portion in the duodenal papilla.

2. The medical support device according to claim 1, wherein the opening portion image includes a first pattern image selected in accordance with a given first instruction from the plurality of first pattern images.

3. The medical support device according to claim 2,
wherein the plurality of first pattern images are displayed one by one as the opening portion image on the screen, and
wherein the first pattern image displayed as the opening portion image on the screen is switched in response to the first instruction.

4. The medical support device according to claim 2, wherein the first geometric feature is a position and/or a size of the opening portion in the duodenal papilla.

5. The medical support device according to claim 1, wherein the opening portion image is an image created based on a first reference image obtained by one or more modalities and/or first information obtained from a medical finding.

6. The medical support device according to claim 1, wherein the opening portion image includes a map indicating a distribution of a probability that the opening portion exists in the duodenal papilla.

7. The medical support device according to claim 6,
wherein the image recognition processing is AI-based image recognition processing, and
wherein the distribution of the probability is obtained by the image recognition processing being executed.

8. The medical support device according to claim 1, wherein a size of the opening portion image changes in accordance with a size of the duodenal papilla region in the screen.

9. The medical support device according to claim 1, wherein the opening portion consists of one or more openings.

10. The medical support device according to claim 1, wherein the processor displays a duct path image indicating a path of one or more ducts that are a bile duct and/or a pancreatic duct in accordance with the duodenal papilla region, in the intestinal wall image displayed on the screen.

11. The medical support device according to claim 10, wherein the duct path image includes a second pattern image selected in accordance with a given second instruction from a plurality of second pattern images expressing different second geometric features of the duct in the intestinal wall.

12. The medical support device according to claim 11,
wherein the plurality of second pattern images are displayed one by one as the duct path image on the screen, and
wherein the second pattern image displayed as the duct path image on the screen is switched in response to the second instruction.

13. The medical support device according to claim 11, wherein the second geometric feature is a position and/or a size of the path in the intestinal wall.

14. The medical support device according to claim 10, wherein the duct path image is an image created based on a second reference image obtained by one or more modalities and/or second information obtained from a medical finding.

15. The medical support device according to claim 10, wherein an image in which the duct path image is included in the intestinal wall image is stored in an external device and/or a medical record.

16. The medical support device according to claim 1, wherein an image in which the opening portion image is included in the duodenal papilla region is stored in an external device and/or a medical record.

17. A medical support device comprising:
a processor,
wherein the processor is configured to:
detect a duodenal papilla region by executing image recognition processing on an intestinal wall image obtained by a camera provided in an endoscopic scope imaging an intestinal wall of a duodenum;
display the intestinal wall image on a screen;
display a duct path image indicating a path of one or more ducts that are a bile duct and/or a pancreatic duct in accordance with the duodenal papilla region, in the intestinal wall image displayed on the screen; and
wherein the duct path image is at least one of a plurality of second pattern images expressing different second geometric features of the duct in the intestinal wall.

18. An endoscope comprising:
the medical support device according to claim 1; and
the endoscopic scope.

19. A medical support method comprising:
detecting a duodenal papilla region by executing image recognition processing on an intestinal wall image obtained by a camera provided in an endoscopic scope imaging an intestinal wall of a duodenum;
displaying the intestinal wall image on a screen;
displaying an opening portion image simulating an opening portion existing in a duodenal papilla, in the duodenal papilla region in the intestinal wall image displayed on the screen; and
wherein the opening portion image is at least one of a plurality of first pattern images expressing different first geometric features of the opening portion in the duodenal papilla.

20. A medical support method comprising:
detecting a duodenal papilla region by executing image recognition processing on an intestinal wall image obtained by a camera provided in an endoscopic scope imaging an intestinal wall of a duodenum;
displaying the intestinal wall image on a screen;
displaying a duct path image indicating a path of one or more ducts that are a bile duct and/or a pancreatic duct in accordance with the duodenal papilla region, in the intestinal wall image displayed on the screen; and
wherein the duct path image is at least one of a plurality of second pattern images expressing different second geometric features of the duct in the intestinal wall.

21. A non-transitory computer-readable storage medium storing a program executable by a computer to execute processing comprising:
detecting a duodenal papilla region by executing image recognition processing on an intestinal wall image obtained by a camera provided in an endoscopic scope imaging an intestinal wall of a duodenum;
displaying the intestinal wall image on a screen;
displaying an opening portion image simulating an opening portion existing in a duodenal papilla, in the duodenal papilla region in the intestinal wall image displayed on the screen; and
wherein the opening portion image is at least one of a plurality of first pattern images expressing different first geometric features of the opening portion in the duodenal papilla.

22. A non-transitory computer-readable storage medium storing a program executable by a computer to execute processing comprising:
detecting a duodenal papilla region by executing image recognition processing on an intestinal wall image obtained by a camera provided in an endoscopic scope imaging an intestinal wall of a duodenum;
displaying the intestinal wall image on a screen;
displaying a duct path image indicating a path of one or more ducts that are a bile duct and/or a pancreatic duct in accordance with the duodenal papilla region, in the intestinal wall image displayed on the screen; and
wherein the duct path image is at least one of a plurality of second pattern images expressing different second geometric features of the duct in the intestinal wall.
